(19) United States
(12) Patent Application Publication
(10) Pub. No.: US 2023/0301514 A1
(43) Pub. Date: Sep. 28, 2023

(54) ULTRASONIC IMPLANT AND SYSTEM FOR MEASUREMENT OF INTRAOCULAR PRESSURE

(71) Applicant: Iota Biosciences, Inc., Alameda, CA (US)

(72) Inventors: Jose LEPE, Martinez, CA (US); Joseph T. GREENSPUN, San Francisco, CA (US); Giana MONTERO GARNIER, Berkeley, CA (US); Michel M. MAHARBIZ, El Cerrito, CA (US); Jose M. CARMENA, Alameda, CA (US); Kunitake ABE, Tokyo (JP); Hiroshi ANDO, Tokyo (JP); Chisato KAMEOKA, Tokyo (JP); Yuki OKUDA, Tokyo (JP); Toshiyuki FUNATSU, Tokyo (JP); Shuhei FUJITA, Tokyo (JP)

(21) Appl. No.: 18/020,344

(22) PCT Filed: Aug. 10, 2021
(86) PCT No.: PCT/US2021/045429

§ 371 Date: Feb. 8, 2023

Related U.S. Application Data
(60) Provisional application No. 63/064,298, filed on Aug. 11, 2020.

(51) Int. Cl. A61B 3/16

(52) U.S. Cl. CPC A61B 3/16; A61B 8/10

(57) ABSTRACT

A device for measuring an intraocular pressure that includes: a pressure sensor configured to measure the intraocular pressure; an ultrasonic transducer electrically coupled to the pressure sensor and configured to receive ultrasonic waves and emit ultrasonic backscatter encoding a pressure measured by the pressure sensor, and a substrate attached to the pressure sensor and the ultrasonic transducer, and configured to interface a surface on or within an eye.

### CROSS-REFERENCE TO RELATED APPLICATION

This application claims the priority benefit to U.S. Provisional Application No. 63/064,298, filed on Aug. 11, 2020, which is incorporated herein by reference for all purposes.

### FIELD OF THE DISCLOSURE

The present invention relates to devices for sensing and reporting eye conditions, such as intraocular pressure, in a subject using ultrasonic backscatter communication.

### BACKGROUND OF THE DISCLOSURE

Intraocular pressure (IOP) of a patient is typically monitored by an eye care professional to assess whether the patient has or is at risk for developing glaucoma. Glaucoma is an eye disease known to cause damage to the optic nerve, resulting in vision loss. The optic nerve can be affected by high IOP and thus early detection of high IOP is typically used to provide early treatment options for minimizing vision loss associated with high IOP. In general, regular monitoring of IOP can help identify abnormal IOP readings based on IOP trends of a patient. A widely accepted method for accurately measuring IOP requires assistance of an eye care professional to administer anesthetic eye drops, fluorescent dye, and measure intraocular pressure using specialized tonometry equipment. The specialized tonometry equipment includes a tip that is used to flatten the cornea of an eye by applying a calibrated amount of force. The reliance on an eye care professional for IOP monitoring limits the frequency of IOP monitoring to the number of patient visits to an eye care professional.

### SUMMARY OF THE DISCLOSURE

Described herein are devices, systems, and methods that allows for on-demand collection of intraocular pressure (IOP) measurements. These devices, systems, and methods may be used outside of a clinical setting, allowing a patient to measure eye pressures more frequently and as desired. Regular use of the on-demand IOP measurement collection can play a key role in monitoring ocular disease progression and allows for fast treatment response times.

In some embodiments, a device for measuring an intraocular pressure, includes: a pressure sensor configured to measure the intraocular pressure; an ultrasonic transducer electrically coupled to the pressure sensor and configured to receive ultrasonic waves and emit ultrasonic backscatter encoding a pressure measured by the pressure sensor; and a substrate attached to the pressure sensor and the ultrasonic transducer, and configured to interface a surface on or within an eye.

In any of these embodiments, the substrate may have a partial or full ring structure. In some embodiments, the substrate is configured to apply a force to the substrate, such as a radial outward force. In some embodiments, the device is configured to be implanted within a capsular bag of the eye. In any of these embodiments, the substrate may include one or more apertures configured to secure a surgical tool for guiding the device during implantation. In any of these embodiments, the device may comprise a housing configured to enclose the pressure sensor and the ultrasonic transducer and interface the substrate. In any of these embodiments, the housing may be mounted on the substrate. In any of these embodiments, the substrate may have a partial or full ring structure, and may include a mount configured to mount the housing. In any of these embodiments, the mount may be configured to extend radially inwardly or radially outwardly from the substrate. In any of these embodiments, the housing may be hermetically sealed. In any of these embodiments, the housing may include an acoustic window. In any of these embodiments, the pressure sensor may be positioned within the housing, and the acoustic window may be configured to equilibrate a pressure inside the housing to a pressure outside the housing. In any of these embodiments, the housing may be filled with a liquid or gel configured to transmit ultrasonic waves. In any of these embodiments, the housing may be filled with silicone oil.

In any of these embodiments, the device may include a temperature sensor. In some embodiments, the device is configured to calibrate the pressure measured by the pressure sensor using an eye temperature measured by the temperature sensor.

In any of these embodiments, the ultrasonic transducer may have a longest length dimension of 1 mm or less.

In any of these embodiments, the surface may include a capsular bag, haptics of an intraocular lens, or a contact lens.

In any of these embodiments, the surface may include an iris.

In any of these embodiments, the surface may include a lens capsule, an episclera, or on or near a pars plana of the eye.

In any of these embodiments, the substrate may include one or more fasteners for attaching the substrate to the surface of the eye. In any of these embodiments, the device may include at least two fasteners positioned at opposite ends of the substrate. In any of these embodiments, the fasteners may include lateral hooks configured to attach to eye tissue. In any of these embodiments, the fasteners may include vertical hooks configured to enter eye tissue.

In any of these embodiments, the ultrasonic transducer may be configured to receive ultrasonic waves that power the implantable device.

In any of these embodiments, the ultrasonic waves may be transmitted by an interrogator external to the device.

In any of these embodiments, the device may comprise an integrated circuit in electrical communication with the pressure sensor and the ultrasonic transducer. In any of these embodiments, the integrated circuit may be configured to power the pressure sensor. In any of these embodiments, wherein the integrated circuit may be configured to encode the measured pressure in the ultrasonic backscatter. In any of these embodiments, the housing may enclose the integrated circuit. In any of these embodiments, the integrated circuit may be coupled to a power circuit comprising a capacitor. In any of these embodiments, the ultrasonic transducer may receive ultrasonic waves that are converted into an electrical energy, which is stored by the power circuit. In any of these embodiments, the integrated circuit may selectively operate the device in a communication mode or power storage mode.

In any of these embodiments, the ultrasonic transducer may be a piezoelectric crystal.

In any of these embodiments, the device may be configured to be implanted within the eye of a subject. In any of these embodiments, the device may be configured to be implanted within an anterior chamber of the eye.

In any of these embodiments, the device may be configured to be battery-less.

In some embodiments, a system for measuring intraocular pressure of an eye, the system includes: the device of any one of these embodiments and an interrogator comprising: a pressure sensor configured to measure ambient pressure; and one or more ultrasonic transducers configured to transmit the ultrasonic waves to implantable device, and receive the ultrasonic backscatter from the implantable device.

In any of these embodiments, the interrogator may be configured to determine the measured intraocular pressure using on the received ultrasonic backscatter. In any of these embodiments, the interrogator may be configured to determine an adjusted intraocular pressure by calibrating the measured intraocular pressure further based on the measured ambient pressure.

In any of these embodiments, the device may include a temperature sensor positioned on the device configured to measure eye temperature. Temperature detected by the device may be used, for example, to calibrate the pressure measurements made by the pressure sensor on the device. In any of these embodiments, the interrogator may be configured to determine the adjusted intraocular pressure by calibrating the measured intraocular pressure based on the measured ambient pressure and measured eye temperature.

In any of these embodiments, the interrogator may include a force gauge configured to measure a force applied by the interrogator. In any of these embodiments, the interrogator may be configured to operate the device to determine a plurality of IOP measurements as the force gauge measures a decreasing force. In any of these embodiments, the interrogator may be configured to select an IOP measurement at a lowest measured force.

In any of these embodiments, the ultrasonic transducer of the interrogator may be configured to transmit ultrasonic waves that power the implantable device.

In some embodiments, a system for measuring intraocular pressure of an eye, comprising an interrogator includes: a pressure sensor configured to measure ambient pressure; and one or more ultrasonic transducers configured to transmit the ultrasonic waves and receive the ultrasonic backscatter encoding an intraocular pressure measured by a device on or in the eye, and wherein the interrogator is configured to determine a measured intraocular pressure based on the received ultrasonic backscatter, and determine an adjusted intraocular pressure by adjusting the measured intraocular pressure based on the measured ambient pressure.

In any of these embodiments, the ultrasonic waves may be configured to power the device.

In any of these embodiments, the ultrasonic waves may be configured to encode instructions for one or more of resetting and the device, designating a mode of operation for the device, setting device parameters for the device, and beginning a data transmission sequence from the device.

In some embodiments, a method of measuring intraocular pressure of an eye, includes: transmitting ultrasonic waves from one or more ultrasonic transducers of an interrogator; receiving the ultrasonic waves transmitted by the one or more ultrasonic transducers of the interrogator at one or more ultrasonic transducers of a device within or on the eye; detecting an intraocular pressure using a pressure sensor on the device; emitting ultrasonic backscatter encoding the intraocular pressure from the ultrasonic transducer of the device; receiving the ultrasonic backscatter at the one or more ultrasonic transducers of the interrogator; determining the measured intraocular pressure from the ultrasonic backscatter; measuring an ambient pressure; and determining an adjusted intraocular pressure by adjusting the measured intraocular pressure based on the measured ambient pressure.

In any of these embodiments, the device may be implanted in a capsular bag of the eye.

In any of these embodiments, the method may include converting energy from the ultrasonic waves into an electrical energy that powers the device.

In any of these embodiments, the method may include instructing the device by the interrogator to execute one or more of resetting the device, designating a mode of operation of the device, setting parameters of the device, and beginning a data transmission sequence from the device.

In any of these embodiments, the pressure detection and measurement may be configured to occur during a time in which no ultrasonic waves are being transmitted.

In any of these embodiments, the method may include coupling the one or more ultrasonic transducers of the interrogator to an eyelid of the eye via a couplant.

In any of these embodiments, the method may include applying a force by the interrogator to contact skin of an eyelid, skin over a brow bone, skin over a nasal bone, or skin over an eye socket, moving the interrogator away from the skin until contact with the skin is lost, and measuring by the interrogator a plurality of force magnitudes while the interrogator is in contact with the skin. In any of these embodiments, the method may include receiving by the interrogator a plurality of intraocular pressure measurements while measuring the plurality force magnitudes. In any of these embodiments, the method may include selecting from the plurality of intraocular pressure measurements a final intraocular pressure associated with a minimal force applied by the interrogator.

In any of these embodiments, the method may include placing the ultrasonic transducer of the interrogator over an eyelid of the eye aiming towards the device.

In any of these embodiments, the method may include placing the ultrasonic transducer of the interrogator over skin of an eyelid, skin over a brow bone, skin over a nasal bone, or skin over eye socket.

In any of these embodiments, the method may include detecting an intraocular eye temperature. In some embodiments, the detected intraocular eye temperature is used to calibrate the intraocular pressure measured by the device. In some embodiments, the intraocular temperature is encoded in the emitted ultrasonic backscatter, and the intraocular pressure detected by the device is calibrated by the interrogator. In some embodiments, the intraocular pressure detected by the device is calibrated by the device.

In some embodiments, a method for treating a patient with an eye disease, includes: measuring an intraocular pressure using a system of any one of these embodiments; determining whether the measured intraocular pressure is above a threshold; and upon determination that the measured intraocular pressure is above the threshold, administering a therapeutic agent to the patient.

In any of these embodiments, the eye disease may be glaucoma or ocular hypertension.

In any of these embodiments, the therapeutic agent may decrease the intraocular pressure.

In any of these embodiments, the threshold may be determined based at least in part on routine measurements of the intraocular pressure.

### BRIEF DESCRIPTION OF THE DRAWINGS

FIG. 1 shows an exemplary schematic of an exemplary system for measuring intraocular pressure.

FIG. 2A shows a schematic of an exemplary device, according to some embodiments.

FIG. 2B shows a schematic of an exemplary device, according to some embodiments.

FIG. 2C illustrates an exploded view of the device of FIG. 2B. The exploded view shows the housing of the device detached from the substrate of the device, according to some embodiments.

FIG. 3A shows an exemplary device having a substrate that includes lateral fasteners, the lateral fasteners are configured in an open position.

FIG. 3B shows an exemplary device having a substrate that includes lateral fasteners, the lateral fasteners are configured in a closed position.

FIG. 4A shows a perspective view of an exemplary device having a substrate that includes vertical fasteners.

FIG. 4B shows a side view of the exemplary device of FIG. 4A.

FIG. 5A shows an exemplary schematic of an exemplary device implanted within an eye.

FIG. 5B shows an exemplary cross-sectional schematic of an exemplary device implanted within an eye at an exemplary location.

FIG. 6A shows an exemplary board assembly for a device, which may be enclosed in a housing.

FIG. 6B shows an exemplary board assembly for a device, which may be enclosed in a housing.

FIG. 7 shows a board assembly for a body of a device that includes two orthogonally positioned ultrasonic transducers.

FIG. 8 shows an interrogator in communication with a device. The interrogator can transmit ultrasonic waves. The device emits an ultrasonic backscatter, which can be modulated by the device to encode information.

FIG. 9A shows an exemplary housing having an acoustic window that may be attached to the top of the housing, and a port that may be used to fill the housing with an acoustically conductive material.

FIG. 9B shows an exploded view of a housing may be configured to house a circuit board.

FIG. 10A shows an exemplary interrogator that can be used with a device.

FIG. 10B shows an exemplary schematic of an exemplary interrogator.

FIG. 11 shows an exemplary interrogator that can be used with a device.

FIG. 12 shows a flowchart of an exemplary method for measuring IOP.

FIG. 13 shows a flowchart of an exemplary method for treating an eye disease.

FIG. 14 shows a flowchart demonstrating a method for using a device for monitoring IOP.

FIG. 15 shows a flowchart demonstrating a method for taking IOP measurements with a device mounted on or within an eye of a patient and an external interrogator.

FIG. 16 shows an example of a computing device according to examples of the disclosure.

### DETAILED DESCRIPTION

The devices disclosed herein are configured for measuring and communicating IOP data. The devices include a substrate, a sensor, and an ultrasonic transducer. The substrate is configured as a platform for mounting the device on or within an eye. The devices are configured to measure IOP data using the sensor and electrically communicate the measured IOP data to the ultrasonic transducer onboard the device.

The systems disclosed herein include a device and an interrogator for measuring and communicating IOP data. The device is configured to be implanted within an eye or mounted on an eye. From its implanted or mounted location, the device is configured to measure IOP data using one or more sensors onboard the device, and communicate the measured IOP data to the interrogator using ultrasonic backscatter communication. The interrogator is configured to receive the measured TOP data, measure environmental conditions, determine a final TOP measurement by adjusting the measured IOP data using the measured environmental conditions, and communicate the final IOP measurement to a recipient external to both the interrogator and the device. The device, the interrogator, and the ultrasonic communication between the device and the interrogator are described further below according to some embodiments.

The devices, systems, and methods disclosed herein enable quick and efficient monitoring of TOP outside a clinical setting, allowing a patient to measure eye pressure frequently and as desired. The capability of measuring eye pressure frequently and as desired enable an on-demand IOP measurement collection towards the prevention and management of glaucoma, ocular hypertension, and/or vision loss associated with abnormal eye pressures. Regular use of on-demand IOP sensing can be used to identify trends in IOP data for early detection of abnormal (high or low) IOP measurements. Furthermore, the dimensions of the device are configured to enable the device to be implanted within an eye via minimally invasive surgery requiring no sutures or mounted on the eye.

### Definitions

As used herein, the singular forms “a,” “an.” and “the” include the plural reference unless the context clearly dictates otherwise.

Reference to “about” a value or parameter herein includes (and describes) variations that are directed to that value or parameter per se. For example, description referring to “about X” includes description of “X”.

The terms “individual,” “patient.” and “subject” are used synonymously, and refer to a mammal.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

In the following description of the disclosure and embodiments, reference is made to the accompanying drawings in which are shown, by way of illustration, specific embodiments that can be practiced. It is to be understood that other embodiments and examples can be practiced, and changes can be made, without departing from the scope of the disclosure.

### Device for Measuring Intraocular Pressure

The device can include a substrate configured to interface a surface on or within the eye. The surface of an eye may include a natural surface of the eye or an engineered surface implanted within or mounted on an eye (such as an intraocular lens implanted within an eye, a phakic intraocular lens implanted within an eye, or a contact lens mounted on an eye). In some embodiments, the substrate can include a flexible material configured to interface with the surface of an eye. In some embodiments, the device can include a housing configured to mount onto the substrate of the device and to house a pressure sensor of the device. The housing can include an acoustic window that allows ultrasonic waves to penetrate and equilibrate pressure external and internal to the housing. The equilibration of pressure enables accurate IOP measurements while protecting the sensor within the housing. The device may include an ultrasonic transducer for receiving the ultrasonic waves penetrating the acoustic window and emitting ultrasonic waves through the acoustic window. In some embodiments, the emitted ultrasonic waves include ultrasonic backscatter configured to be received at a device external to the device.

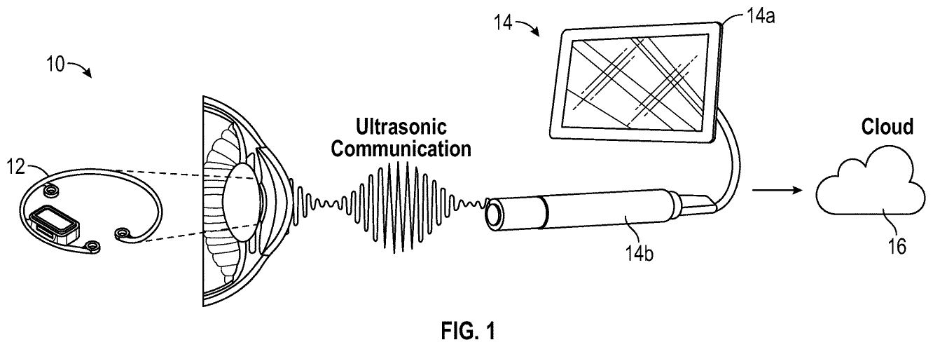

FIG. 1 shows an exemplary schematic of an exemplary system 10 for measuring IOP, according to some embodiments. The system 10 may be configured to monitor IOP in at least two types of patients: those with early-to-late open-angle glaucoma who require regular IOP monitoring and, patients with normal-tension glaucoma with visual field loss who require frequent IOP monitoring. Users of the system may include surgeons implanting or mounting the device, clinicians training and assisting patients in taking IOP measurements, and the patients. In some embodiments, the system 10 may be used in a controlled clinical environment where the clinician can supervise the patient using the system 10. In some embodiments, the system 10 may be used outside a clinical environment, for example in a patient's home.

In some embodiments, the system 10 may include a device 12 and an ultrasonic interrogator 14. The interrogator 14 may include a computer or graphical display 14a configured to process and display IOP data and a head 14b configured to ultrasonically couple to the implanted device 12. In FIG. 1, the device is implanted inside the lens capsule (i.e., capsular bag) of the patient. In other embodiments, the implantable device may interface with and/or be mounted on another surface on or within the eye. The implanted device 12 may measure intraocular pressure data and communicate the measured data to the interrogator 14. The interrogator 14 may process the received measured data before communicating a final IOP measurement to a user.

Optionally, the interrogator 14 can include an application configured to receive processed data from a cloud backend application 16, supply information to a graphical user interface 14a, and enable limited interactions with the ultrasonic interrogator 14. The cloud backend application 16 may be used for data aggregation and analytics.

In some embodiments, a system for measuring IOP may include a plurality of operating states. For example the system 10 may include an OFF, Ready, Search, Measurement Collection, Calibration, Complete, or Inactive or Fault state. In the OFF state, all system components may be powered OFF. In the Ready state, the interrogator 14 may be powered on without active ultrasound. In the Ready state, the interrogator 14 may wait for a user command to start ultrasound transmission. In the Search state, the interrogator 14 may search for, find, and power the device 12. In the Measurement Collection state, the interrogator may query the device 12 for data and perform the measurement calculation, while continuing to power the device. In the Measurement Calibration state, the interrogator may perform calibration of the pressure measurement. In the Measurement Complete state, the interrogator may notify the user that the measurement is complete via the physical and graphical user interfaces. In some embodiments, measurement data may be displayed to the user via a display 14a. In the Inactive or Fault state, an internal interrogator diagnostics may detect a fault and shut down the ultrasonic power while the interrogator remains on. The Inactive or Fault state is different from the Ready state because the ultrasound will not be able to be turned on by the user until the systems returns to the Ready state. This may be the case when there is a system fault sensed or when the interrogator deliberately limits ultrasound power output.

In some embodiments, the system 10 may be configured to receive a manual selection from the user to change to a state where ultrasound power output is active. In some embodiments, the system 10 may automatically stop ultrasound output when the IOP measurement is complete.

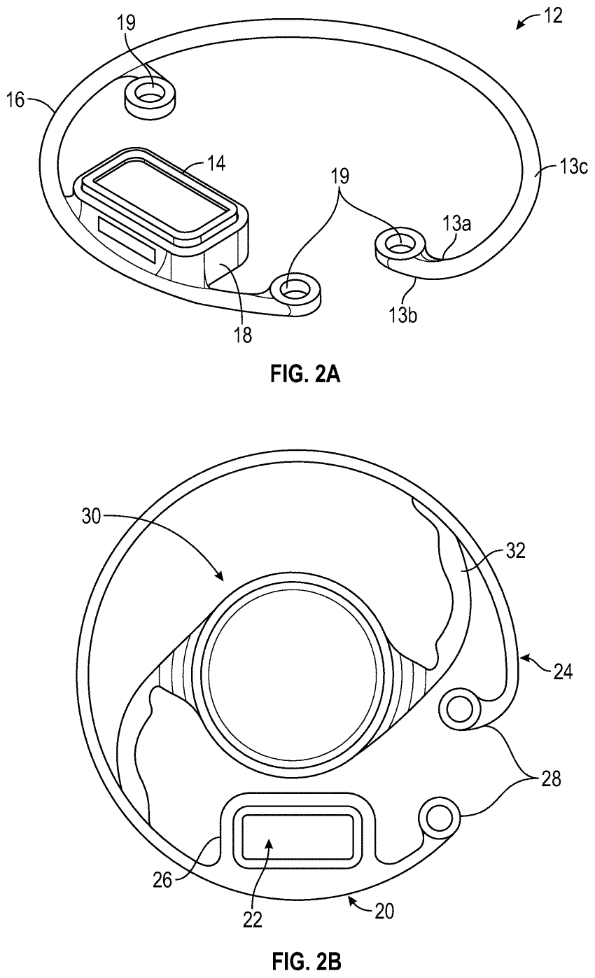

FIG. 2A shows an exemplary schematic of an exemplary device 12, according to some embodiments. The device 12 may be part of an IOP measuring system as shown in system 10. In some embodiments, the device 12 may include a housing 14 that encloses internal components and the housing 14 may be hermetically sealed. In some embodiments, the device 12 may include a substrate 16 configured to attach to and support the housing 14.

In some embodiments, the substrate 16 may be an annular member 16 made of a flexible material. In some embodiments, the substrate 16 may be an annular member 16 configured as a tension ring. The annular member 16 may be configured to exert a radially outward force applied to the interfacing surface. For example, the annular member 16 may be compressed during implantation, generating an outward spring force when relaxed after implantation. The resulting outward force exerted by the annular member 16 can help stabilize the device in position after implantation. In some embodiments, the annular member 16 can be made of polymethylmethacrylate (PMMA). In some embodiments, the annular member 16 may have a full or partial ring structure. In some embodiments, annular member 16 can form at least 50%, at least 60%, at least 70%, at least 80%, at least 90%, or at least 95% of a circle, or a complete circle.

In some embodiments, the ring structure may include a mount (e.g., an inwardly extending portion) 18 configured to mount the housing 14. The mount 18 on the exemplary device sown in FIG. 2A extends inwardly, although in other configurations the mount may extend outwardly or may be positioned on top of the annular member 16. In some embodiments, the size of the annular member 16 may be configured for a particular range of patient eye size. The annular member 16 may include a plurality of apertures 19 that can be used to guide positioning of the device 12 during implantation or mounting. In some embodiments, for an annular member having a partial ring structure, each aperture 19 may be located at an end of the partial ring structure. In some embodiments, one or more of the apertures 19 may be spaced away from an end of the partial ring structure. The apertures 19 may be engaged by an external medical tool (such as a hook, forceps, etc.) for placing the device 12 properly within the eye. In some embodiments, the device 20 may include a top face 13a, a bottom face 13b, and a side face 13c.

FIG. 2B shows a schematic of an exemplary device 20, according to some embodiments. Device 20 may be part of an IOP measuring system such as system 10. Similar to device 12, device 20 may include a housing 22, a substrate 24, an inwardly extending portion 26, and a plurality of apertures 28. FIG. 2B shows the device 20 interfaces with (e.g., may be mounted about) an intraocular lens 30. When implanted within an eye, the intraocular lens 30 may be a surface within the eye.

In some embodiments, the device 20 may be implanted in one of the patient's eyes during the same surgery for intraocular lens placement. In some embodiments, the device 20 may allow co-placement with an intraocular lens. An example of co-placement of device 20 and an intraocular lens 30 is shown in FIG. 2B. In some embodiments, the device 20 may be co-placed with an intraocular lens (e.g., a commercially available intraocular lens) such that the substrate of the device 20 interfaces the arms (e.g., haptics 32) of the intraocular lens 30. The annular member 24 can exert a radial outward force against the haptics 32 of the intraocular lens 30, which stabilizes the device 20 in position. When the annular member 24 is co-placed with an intraocular lens, the placement of the annular member 24 does not interfere with the line of sight of the eye or the functioning of the intraocular lens. In some embodiments, the housing 22, the substrate 24, and the plurality of apertures 28 may be configured to not interfere with the haptics 32 of an intraocular lens 30.

In some embodiments, the device 20 may be co-placed with an intraocular lens such that the top face 13a of the device 20 interfaces the intraocular lens 30. In some embodiments, the device 20 may be co-placed with an intraocular lens such that the bottom face 13b of the device 20 interfaces the intraocular lens 30. In some embodiments, the device 20 may be co-placed with an intraocular lens such that the side face 13c of the device 20 interfaces the intraocular lens 30. In some embodiments, the device 20 may be co-placed with an intraocular lens such that the device interfaces with the haptics of the intraocular lens without interfering with the function of the haptics. In other embodiments, the device 20 may be implanted within other areas of the eye such the posterior chamber and anterior chamber of the eye. The device 20 may be configured to maintain functional integrity as an implanted device for at least about 3 years, 4 years, 5 years, 6 years, 7 years, or more.

FIG. 2C illustrates an exploded view of device 20, according to some embodiments. The exploded view shows the housing 22 detached from the substrate 24, according to some embodiments. As shown in FIG. 2C, the housing 22 may include one or more mounting features 23 (e.g., snaps, clips, outwardly projecting members, etc.) to secure the housing 22 to a mount 34 positioned on the substrate 24 via corresponding features 25 (e.g., receiving snaps, inwardly projecting members, etc.). In some embodiments, the corresponding features 25 may be part of a radially extending portion configured to mount the housing. In some embodiments, the radially extending portion may include side walls 27 configured to at least partially cover side walls 29 of the device 20. In some embodiments, a bottom surface 31 of the device 20 may be configured to interface a surface of the eye when the housing 22 is mounted on the substrate 24 that interfaces with (e.g., is mounted on) the surface on or within the eye, such as an intraocular lens.

In some embodiments, the substrate 24 may be an annular member. In some embodiments, the substrate 24 may be an annular member that is a tension ring. In some embodiments, when the device 20 is implanted within the capsular bag of an eye, the annular member 24 may be configured to apply a supporting force (i.e., a tension) to the capsular bag. In some embodiments, the supporting force may be enough to hold the tension ring in place within the eye. In some embodiments, the annular member 24 may be held in place within the eye and retain its shape based on its size and position within the eye within the capsular bag of the eye. In some embodiments, the annular member 24 may interface a perimeter of the capsular bag.

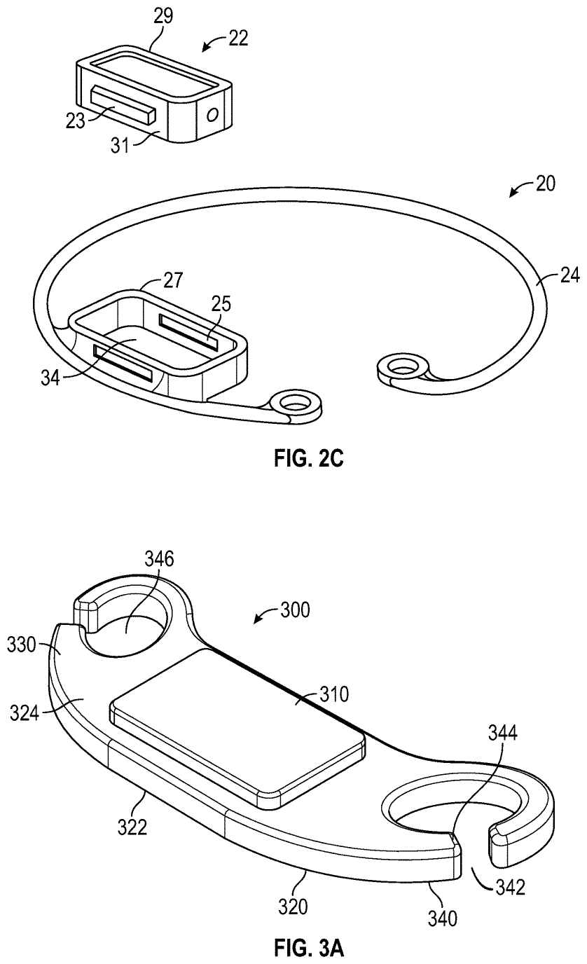

In some embodiments, the substrate may include fasteners to mount the substrate to the surface within the eye. In some embodiments, the fasteners may include a plurality of lateral clamps. FIGS. 3A and 3B show exemplary devices 300, 400, having a respective housing 310, 410 mounted onto a substrate 320, 420, according to some embodiments. The substrate may have a first side for mounting the substrate to a surface within or an eye. For example, FIG. 3A shows substrate 320 having a first side 322 for mounting within or an eye. The surface within the eye may be, for example, an iris, a lens capsule, an episclera, an intraocular lens implanted within an eye, or a phakic intraocular lens implanted within an eye. The substrate 320, 420 can include lateral clamps. A first lateral clamp 330, 430 can be positioned at one end of the substrate 320, 420 and a second lateral clamp 340, 440 can be positioned at an opposite end of the substrate 320, 420. Each lateral clamp may be shaped by a slit in the substrate and may include an open position in which eye tissue of the surface (such as iris 130) within the eye is positioned within the slit and a closed position in which the eye tissue positioned between the slit is clamped to mount the substrate to the surface within the eye. In some embodiments, the slit may be at least about 0.1, 0.2 mm, or 0.4 mm. In some embodiments, the slit may be at most about 1 mm, 0.8 mm, or 0.6 mm. In some embodiments, the slit may be about 0.1-1 mm, 0.2-0.8, or 0.4-0.6 mm.

FIG. 3A shows an example of the lateral clamps 330, 340 in an open position in which eye tissue (such as iris tissue 130) or outermost part of a surface may be positioned within slit 342 in the substrate 320, according to some embodiments. In some embodiments, the device 300 may be configured such that during placement of the device 300, a surgeon may move slit walls 344 to clamp onto eye tissue (such as iris tissue 130) within the slit 342. FIG. 3B shows an example of the lateral clamps 430, 440 in a position in which eye tissue or outermost part of a surface may be clamped within a thinner slit 442 (thinner compared, for example, to the slit 342), according to some embodiments. In some embodiments, the device 400 may be configured such that during placement of the device 400, a surgeon may pinch eye tissue (such as iris tissue 130) to feed the pinched eye tissue through the thinner slit 342. In some embodiments, the lateral clamps may be made from polymer. In some embodiments, the positioning slits of slits 342, 442 may be configured to follow the radial grain of the iris fibers 130

In some embodiments, each slit includes slit walls that are spaced from each other in the open position and the slit walls are movable towards each other for clamping eye tissue in a closed position. For example, slit wall 344 of slit 342 may be configured to clamp onto eye tissue. In some embodiments, the lateral clamps are configured to move from the open position (such as the open position of FIG. 3A) to a closed position by a force applied during a surgical implantation or procedure. The lateral clamps may remain in the closed position until purposefully moved to an open position by a force applied during a surgical procedure. In some embodiments, each slit may extend into a circular aperture (such as aperture 346) of the substrate.

In some embodiments, the substrate may be flexible and may be bonded to the rigid housing. In some embodiments, the housing may attach to the substrate by being fixed on an outer surface of the substrate. In some embodiments, the housing may attach to the substrate by extending through substrate. In some embodiments, the substrate may have a second side for attaching a mountable side of the housing to the substrate. For example, FIG. 3A shows the substrate having a second side 324 on which the housing 310 is mounted.

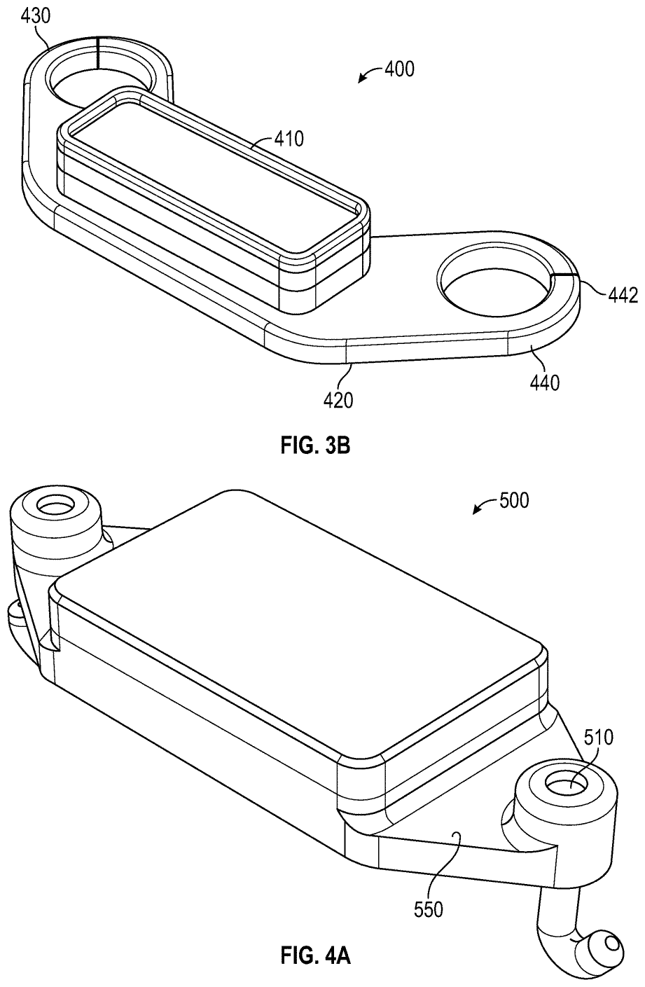

In some embodiments, the fasteners may include a plurality of vertical hooks. FIGS. 4A and 4B show an example of an exemplary device 500 mounted onto a substrate 550 having vertical hooks, according to some embodiments. In some embodiments, the vertical hooks may be insert molded. A first vertical hook 552 may be positioned at the one end of the substrate 550 and a second vertical hook 554 may be positioned at the opposite end of the substrate 550. Each vertical hook may be configured to extend from an interior channel 510 of the substrate 550 that holds a first portion of the vertical hook within the substrate 550. A second portion of each vertical hook may extend in a first direction passed the first side 556 of the substrate and away from the first side 556 of the substrate 550. The second portion of each hook may include an end that extends in a second direction, different from the first direction to form a hook shape. For example, hook 554 can include an end 558 configured to catch eye tissue. Each vertical hook having a hook shape may be configured to enter eye tissue for mounting the substrate to the surface within the eye. For example, the hooks 552, 554 are configured to pass through the tissue of an eye surface (such as iris surface 130) to mount the device 500 on the eye surface. When the hooks 552, 554 pass through eye tissue or outermost part of the eye surface, the hooks 552, 554 are configured to prevent the device 50) from being unmounted from the eye surface. In some embodiments, the vertical hooks 552, 554 may be pushed towards the surface within the eye to insert the vertical hooks 552, 554 within the eye tissue. In some embodiments, the vertical hooks may be made from polymer.

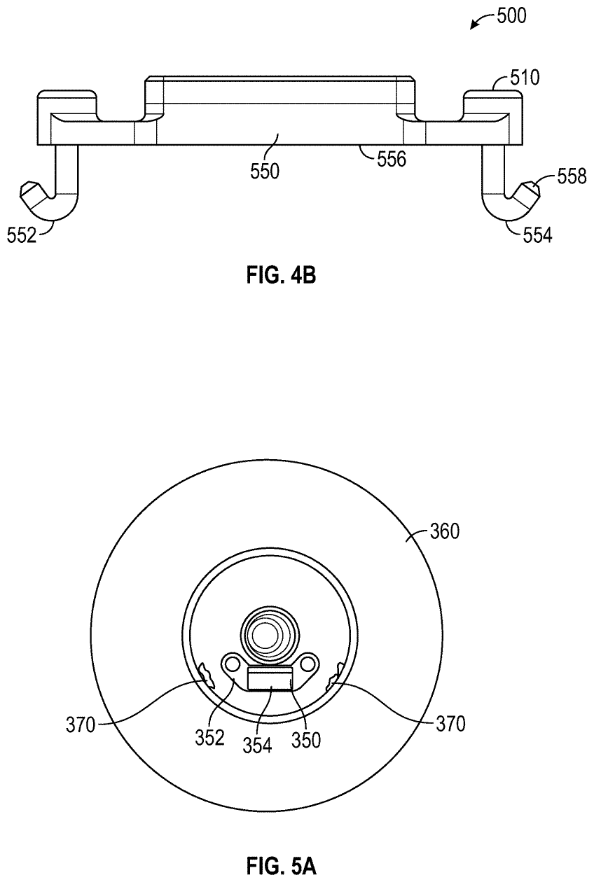

FIG. 5A and FIG. 5B show a schematic of an exemplary device 350 (such as devices 300, 400) having an exemplary substrate 352 (such as 320, 420) for mounting the device 350 within an eye 360 and an exemplary housing 354 (such as 310, 410) for housing internal components of the device, according to some embodiments. FIG. 5A shows an exemplary top-view of the device 350 mounted within the eye 360, according to some embodiments. In other embodiments, the device 350 may be configured to be mounted on an eye. The device 350 may be configured to maintain functional integrity as a mounted or implanted device for at least about 3 years, 4 years, 5 years, 6 years, 7 years, or more.

FIG. 5A shows possible exemplary locations for minimally invasive incision sites 370 for mounting the device 350 within the eye 360 such that mounted device does not interfere with the line of sight of the eye 360. FIG. 5B shows an exemplary cross-sectional schematic displaying the exemplary device 350 mounted to a surface 380 within the eye 360, according to some embodiments. As shown in FIG. 5B, the surface 380 within the eye 360 may be a top surface of the iris located in anterior chamber of an eye. Mounting the device on the top surface of the iris located in the anterior chamber as shown in FIG. 5B, rather than mounting on a bottom surface of the iris located in the posterior chamber of the eye, is advantageous because there is less risk of damaging the iris during implantation compared to mounting on the bottom surface of the iris. In some embodiments, the surface within the eye may be on or near a pars plana 382 of the ciliary body of the eye.

In other embodiments, the device may be implanted within the capsular bag. For example, the device may be co-placed with an intraocular lens.

The device is configured to measure IOP data and encode IOP data via ultrasonic backscatter using internal components of the device, such as one or more sensors, one or more transducers, and an integrated circuit. Exemplary implantable devices that are powered by ultrasonic waves and can emit an ultrasonic backscatter encoding a detected physiological condition are described in WO 2018/009905 and WO 2018/009911.

An integrated circuit of the device can electrically connect and communicate with the one or more sensors of the device and the wireless communication system (e.g., the one or more ultrasonic transducers). The integrated circuit can include or operate a modulation circuit within the wireless communication system, which modulates an electrical current flowing through the wireless communication system (e.g., one or more ultrasonic transducers) to encode information in the electrical current. The modulated electrical current affects backscatter waves (e.g., ultrasonic backscatter waves) emitted by the wireless communication system, and the backscatter waves encode the information.

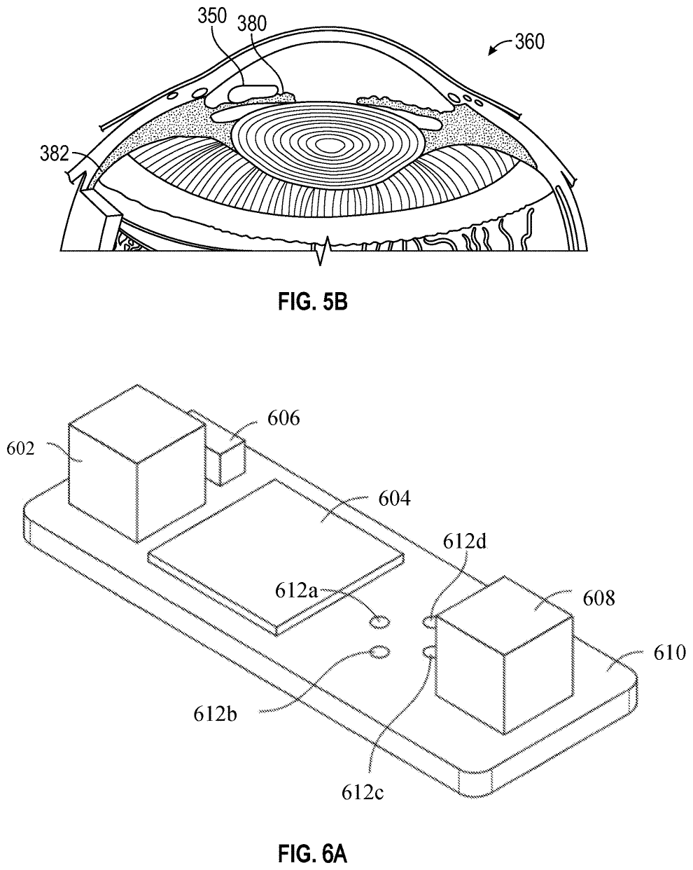

FIG. 6A shows a side view of an exemplary board assembly of an exemplary device, which may be surrounded by a housing (such as housing 14, 22, 310, or 410) and include an integrated circuit, according to some embodiments. The device includes a wireless communication system (e.g., one or more ultrasonic transducers) 602 and an integrated circuit 604. In the illustrated embodiment, the integrated circuit 604 includes a power circuit that includes a capacitor 606. In the illustrated embodiment, the capacitor is an “off chip” capacitor (in that it is not on the integrated circuit chip), but is still electrically integrated into the circuit. The capacitor can temporarily store electrical energy converted from energy (e.g., ultrasonic waves) received by the wireless communication system, and can be operated by the integrated circuit 604 to store or release energy. The device further includes one or more sensors 608. The one or more sensors can include a pressure sensor. Since ultrasound waves transmitted to and from the device may affect sensor measurements, the one or more sensors of the device may be configured to measure IOP data when ultrasound waves are not being transmitted. The one or more ultrasonic transducers 602, integrated circuit 604, the capacitor 606, and the one or more sensors 608 are mounted on a circuit board 610, which may be a printed circuit board. In some embodiments, the one or more ultrasonic transducers 602, integrated circuit 604, the capacitor 606, and the one or more sensors 608 are adhered on the circuit board 610. In some embodiments, the circuit board 610 may include ports 612a-d. Similar to FIG. 6A, FIG. 6B shows a side view of an exemplary board assembly that may be enclosed in a housing, according to some embodiments. The board assembly of FIG. 6B includes a piezoelectric transducer 602b and one or more sensors 608b adhered on the circuit board 610b, according to some embodiments.

The wireless communication system of the device can be configured to receive instructions for operating the device. The instructions may be transmitted, for example, by a separate device, such as an interrogator. By way of example, ultrasonic waves received by the device (for example, those transmitted by the interrogator) can encode instructions for operating the device. The instructions may include, for example, a trigger signal that instructs the device to operate the pressure sensor to detect the intraocular pressure.

An interrogator can transmit energy waves (e.g., ultrasonic waves), which are received by the wireless communication system of the device to generate an electrical current flowing through the wireless communication system (e.g., to generate an electrical current flowing through the ultrasonic transducer). The flowing current can then generate backscatter waves that are emitted by the wireless communication system. The modulation circuit can be configured to modulate the current flowing through the wireless communication system to encode the information. For example, the modulation circuit may be electrically connected to an ultrasonic transducer, which received ultrasonic waves from an interrogator. The current generated by the received ultrasonic waves can be modulated using the modulation circuit to encode the information, which results in ultrasonic backscatter waves emitted by the ultrasonic transducer to encode the information. The modulation circuit includes one or more switches, such as an on/off switch or a field-effect transistor (FET). An exemplary FET that can be used with some embodiments of the implantable device is a metal-oxide-semiconductor field-effect transistor (MOSFET). The modulation circuit can alter the impedance of a current flowing through the wireless communication system, and variation in current flowing through the wireless communication system encodes the information. In some embodiments, information encoded in the backscatter waves includes information related to an electrical pulse emitted by the device, or a physiological condition detected by the one or more sensors of the device. In some embodiments, information encoded in the backscatter waves includes a unique identifier for the device. This can be useful, for example, to ensure the interrogator is in communication with the correct implantable device when a plurality of implantable devices is implanted in the subject. In some embodiments, the information encoded in the backscatter waves includes a verification signal that verifies an electrical pulse was emitted by the device. In some embodiments, the information encoded in the backscatter waves includes an amount of energy stored or a voltage in the energy storage circuit (or one or more capacitors in the energy storage circuit). In some embodiments, the information encoded in the backscatter waves includes a detected impedance. Changes in the impedance measurement can identify scarring tissue or degradation of the electrodes over time.

In some embodiments, the modulation circuit is operated using a digital circuit or a mixed-signal integrated circuit (which may be part of the integrated circuit), which can actively encode the information in a digitized or analog signal. The digital circuit or mixed-signal integrated circuit may include a memory and one or more circuit blocks, systems, or processors for operating the implantable device. These systems can include, for example, an onboard microcontroller or processor, a finite state machine implementation, or digital circuits capable of executing one or more programs stored on the implant or provided via ultrasonic communication between interrogator and implantable device. In some embodiments, the digital circuit or a mixed-signal integrated circuit includes an analog-to-digital converter (ADC), which can convert analog signal encoded in the ultrasonic waves emitted from the interrogator so that the signal can be processed by the digital circuit or the mixed-signal integrated circuit. The digital circuit or mixed-signal integrated circuit can also operate the power circuit, for example to generate the electrical pulse to operate the pressure sensor to detect IOP. In some embodiments, the digital circuit or the mixed signal integrated circuit receives the trigger signal encoded in the ultrasonic waves transmitted by the interrogator, and operates the power circuit to discharge the electrical pulse in response to the trigger signal.

In some embodiments, the one or more sensors 608 may a pressure sensor configured to measure IOP. The pressure sensor may implement capacitive or resistive pressure sensing. The measurement accuracy of the pressure sensor may be at least 0.1 mmHg, 0.2 mmHg, 0.3 mmHg, 0.4 mmHg, or 0.5 mmHg. The measurement accuracy of the pressure sensor may be at most 1.0 mmHg, 0.9 mmHg, 0.8 mmHg, 0.6 mmHg, or 0.7 mmHg. The measurement accuracy of the pressure sensor may be 0.1-1.0 mm Hg, 0.2-0.9 mm Hg, 0.3-0.8 mm Hg, 0.4-0.7 mm Hg. or 0.5-0.6 mmHg. In some embodiments, the measurement accuracy of the pressure sensor may be over a range of 1 mmHg to 70 mmHg, 3 mmHg to 60 mmHg, or 5 mmHg to 50 mmHg. In some embodiments, the pressure sensor may have a sensitivity of about 10 μV/V/mmHg, 20 μV/V/mmHg, or 30 μV/V/mmHg. In some embodiments, the pressure sensor may have a sensitivity requirement dependent on the sensitivity of the readout electronics. In some embodiments, the pressure sensor may have a measurement accuracy and sensitivity range dependent on the sensitivity of the readout electronics.

In some embodiments, the pressure sensor may be temperature sensitive. The pressure sensor may be calibrated based on a temperature response of the temperature sensor. The calibration may be configured to ensure that a difference in pressure output of the pressure sensor is an actual different in pressure and not an artifact of a change in temperature.

In some embodiments, the one or more sensors may include a temperature sensor configured to measure an anterior chamber temperature of an eye. In some embodiments, the temperature sensor may have an accuracy of about 0.1-1° C., 0.2-0.8° C., or 0.3-0.6° C. In some embodiments, the temperature sensor may monitor a range of temperature inside the eye from about 28° C. to 46° C., 30° C. to 44° C., or 32° C. to 40° C. In some embodiments, the temperature sensor data may be used for compensation purposes to increase accuracy of the final pressure measurement.

Both the pressure data from the pressure sensor and temperature data from the temperature sensor may be reported to the external interrogator. The reported pressure data and the reported temperature data may be an averaged or processed result taken from multiple discrete measurements from the corresponding sensor. In some embodiments, the temperature measurement is used to calibrate the measured pressure at the device, and the ultrasonic backscatter can communicate a calibrated pressure. In some embodiments, the pressure data reported by the device may be equivalent to pressure outside of the device with a lag of no more than 1 second, 3 seconds, or 5 seconds. In some embodiments, the time from when the measurement command is received from the external interrogator to when the measurement is reported to the interrogator shall be no more than 2 seconds, 4 seconds, 6 seconds, or 8 seconds.

In some embodiments, the wireless communication system includes one ultrasonic transducer that is an ultrasonic transceiver configured to convert mechanical energy from ultrasound waves to electrical current and vice versa. The ultrasonic transducer may be capable of harvesting energy originating from an external ultrasonic interrogator and capable of producing a modulation depth detectable by an external interrogator.

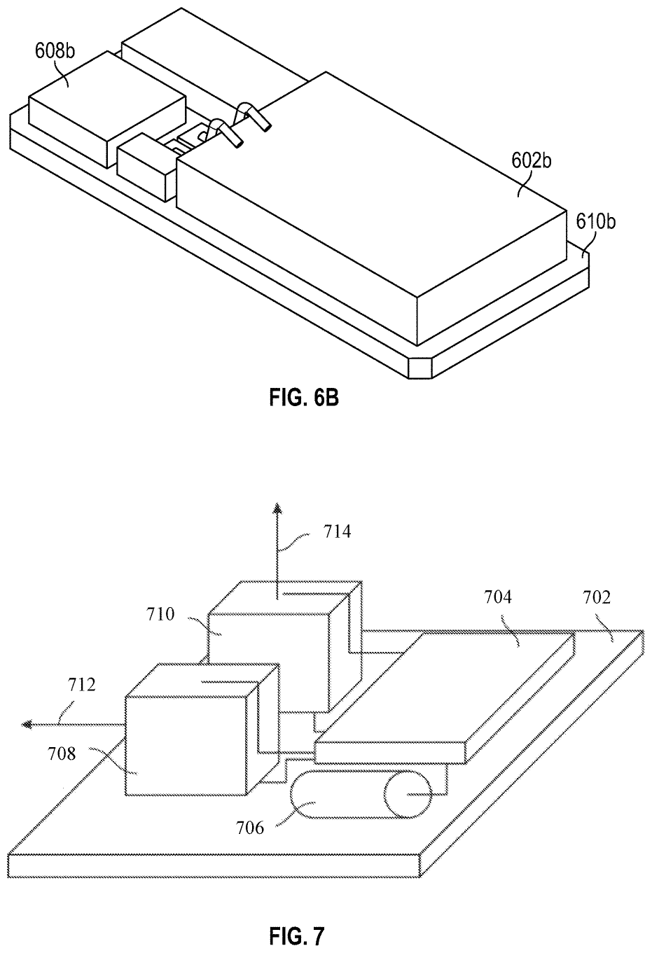

In some embodiments, the wireless communication system includes one or more ultrasonic transducers, such as one, two, or three or more ultrasonic transducers. In some embodiments, the wireless communication system includes a first ultrasonic transducer having a first polarization axis and a second ultrasonic transducer having a second polarization axis, wherein the second ultrasonic transducer is positioned so that the second polarization axis is orthogonal to the first polarization axis, and wherein the first ultrasonic transducer and the second ultrasonic transducer are configured to receive ultrasonic waves that power the device and emit an ultrasonic backscatter. In some embodiments, the wireless communication system includes a first ultrasonic transducer having a first polarization axis, a second ultrasonic transducer having a second polarization axis, and a third ultrasonic transducer having a third polarization axis, wherein the second ultrasonic transducer is positioned so that the second polarization axis is orthogonal to the first polarization axis and the third polarization axis, wherein the third ultrasonic transducer is positioned so that the third polarization axis is orthogonal to the first polarization and the second polarization axis, and wherein the first ultrasonic transducer and the second ultrasonic transducer are configured to receive ultrasonic waves that power the device and emit an ultrasonic backscatter. FIG. 7 shows a board assembly of a device that includes two orthogonally positioned ultrasonic transducers. The device includes a circuit board 702, such as a printed circuit board, and an integrated circuit 704, which a power circuit that includes a capacitor 706. The device further includes a first ultrasonic transducer 708 electrically connected to the integrated circuit 704, and a second ultrasonic transducer 710 electrically connected to the integrated circuit 704. The first ultrasonic transducer 708 includes a first polarization axis 712, and the second ultrasonic transducer 710 includes a second polarization axis 714. The first ultrasonic transducer 708 and the second ultrasonic transducer are positioned such that the first polarization axis 712 is orthogonal to the second polarization axis 714.

The one or more ultrasonic transducers, if included in the wireless communication system, can be a micro-machined ultrasonic transducer, such as a capacitive micro-machined ultrasonic transducer (CMUT) or a piezoelectric micro-machined ultrasonic transducer (PMUT), or can be a bulk piezoelectric transducer. Bulk piezoelectric transducers can be any natural or synthetic material, such as a crystal, ceramic, or polymer. Exemplary bulk piezoelectric transducer materials include barium titanate (BaTiO₃), lead zirconate titanate (PZT), zinc oxide (ZO), aluminum nitride (AlN), quartz, berlinite (AlPO₄), topaz, langasite (La₃Ga₅SiO₁₄), gallium orthophosphate (GaPO₄), lithium niobate (LiNbO₃), lithium tantalite (LiTaO₃), potassium niobate (KNbO₃), sodium tungstate (Na₂WO₃), bismuth ferrite (BiFeO₃), polyvinylidene (di)fluoride (PVDF), and lead magnesium niobate-lead titanate (PMN-PT).

In some embodiments, the bulk piezoelectric transducer is approximately cubic (i.e., an aspect ratio of about 1:1:1 (length:width:height). In some embodiments, the piezoelectric transducer is plate-like, with an aspect ratio of about 5:5:1 or greater in either the length or width aspect, such as about 7:5:1 or greater, or about 10:10:1 or greater. In some embodiments, the bulk piezoelectric transducer is long and narrow, with an aspect ratio of about 3:1:1 or greater, and where the longest dimension is aligned to the direction of the ultrasonic backscatter waves (i.e., the polarization axis).

In some embodiments, one dimension of the bulk piezoelectric transducer is equal to one half of the wavelength (λ) corresponding to the drive frequency or resonant frequency of the transducer. At the resonant frequency, the ultrasound wave impinging on either the face of the transducer will undergo a 180° phase shift to reach the opposite phase, causing the largest displacement between the two faces. In some embodiments, the piezoelectric crystal may be assembled into the housing such that its poled direction is perpendicular to an acoustic window.

In some embodiments, the height of the piezoelectric transducer is about 10 μm to about 1000 μm (such as about 40 μm to about 400 μm, about 100 μm to about 250 μm, about 250 μm to about 500 μm, or about 500 μm to about 1000 μm). In some embodiments, the height of the piezoelectric transducer is about 5 mm or less (such as about 4 mm or less, about 3 mm or less, about 2 mm or less, about 1 mm or less, about 500 μm or less, about 400 μm or less, 250 μm or less, about 100 μm or less, or about 40 μm or less). In some embodiments, the height of the piezoelectric transducer is about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in length. In some embodiments, the ultrasonic transducer has a length of about 5 mm or less such as about 4 mm or less, about 3 mm or less, about 2 mm or less, about 1 mm or less, about 500 μm or less, about 400 μm or less, 250 μm or less, about 100 μm or less, or about 40 μm or less) in the longest dimension. In some embodiments, the ultrasonic transducer has a length of about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in the longest dimension.

In some embodiments the micro-machined piezoelectric crystal can have dimensions of about at least 0.3 micrometer×0.3 micrometer×0.1 micrometer. In some embodiments, the piezoelectric crystal can have dimensions of about at most 1.2 micrometer×1.2 micrometer×0.6 micrometer. In some embodiments, the piezoelectric crystal can have dimensions of about 0.3-1.2 micrometer×0.3-1.2 micrometer×0.1-0.6 micrometer.

The one or more ultrasonic transducers, if included in the wireless communication system, can be connected to two electrodes to allow electrical communication with the integrated circuit. The first electrode is attached to a first face of the transducer and the second electrode is attached to a second face of the transducer, wherein the first face and the second face are opposite sides of the transducer along one dimension. In some embodiments, the electrodes comprise silver, gold, platinum, platinum-black, poly(3,4-ethylenedioxythiophene (PEDOT), a conductive polymer (such as conductive PDMS or polyimide), or nickel. In some embodiments, the axis between the electrodes of the transducer is orthogonal to the motion of the transducer.

The wireless communication system may be used to wireless receive the energy, or a separate system may be configured to receive the energy. For example, an ultrasonic transducer (which may be an ultrasonic transducer contained within the wireless communication system or a different ultrasonic transducer) can be configured to receive ultrasonic waves and convert energy from the ultrasonic waves into an electrical energy. The electrical energy is transmitted to the integrated circuit to power the device. The electrical energy may power the device directly, or the integrated circuit may operate a power circuit to store the energy for later use.

In some embodiments, the integrated circuit may be configured to control the harvesting of energy from the received ultrasonic waves, power the one or more sensors, and encode the eye-related data collected by the one or more sensors using backscatter modulation. The encoding of the eye-related data includes digitizing the eye-related data collected by the one or more sensors and modulating the characteristics of electrical current within the device for digital backscatter communication with the external interrogator. In some embodiments, the integrated circuit (such as integrated circuit 604, 704) is an application specific integrated circuit (ASIC). In some embodiments, the ASIC operation may be passive. The ASIC may power up and transmit messages only when commanded by the external interrogator. In some embodiments, there is no OFF command for the ASIC since the ASIC may be powered off by stopping ultrasound communication between the device and the external interrogator. The stopping of the ultrasound communication may quickly deplete the energy store of the device. When powered, the ASIC may transmit data bits or acknowledgments to the interrogator to allow for status evaluation of the ultrasound communication link. When a measurement command is received the ASIC may perform the command if it can complete the command with the available power.

In some embodiments, power may be harvested from the received ultrasonic waves using the piezoelectric crystal of the ultrasonic transducer and the ASIC of the device. The ASIC may convert AC ultrasonic power to DC power, may be able to sustain operation of the device with a minimum average power, and may generate an IOP measurement within a pre-determined amount of time. In some embodiments, the minimum average power may be about 10×10⁻⁶ W, 20×10⁻⁶ W, or 30×10⁻⁶ W average power. In some embodiments, the pre-determined amount of time may be about less than 1 second, 3 seconds, or 5 second.

In some embodiments, the integrated circuit includes a power circuit, which can include an energy storage circuit. The energy storage circuit may include a battery, or an alternative energy storage device such as one or more capacitors. The device may be batteryless, and may rely on one or more capacitors. By way of example, energy from ultrasonic waves received by the device (for example, through the wireless communication system) is converted into a current, and can be stored in the energy storage circuit. The energy can be used to operate the device, such as providing power to the digital circuit, the modulation circuit, or one or more amplifiers, or can be used to generate an electrical pulse. In some embodiments, the power circuit further includes, for example, a rectifier and/or a charge pump.

In some embodiments, the piezoelectric crystal may be electrically and mechanically connected to the ASIC and substrate such that the Curie temperature, the resonant frequency, and resistance range at resonance are maintained within pre-determined ranges. In some embodiments, the Curie temperature may be at least about 180° C., 200° C., or 220° C. In some embodiments, the Curie temperature may be at most about 260° C., 250° C., or 240° C. In some embodiments, the Curie temperature may be about 180 to 60° C., 200 to 250° C., or 220 to 240° C. In some embodiments, the resonant frequency may be at least about 1.2 MHz, 1.4 MHz, 1.6 MHz, or 1.8 MHz. In some embodiments, the resonant frequency may be at most about 2.8 MHz, 2.6 MHz, 2.4 MHz, or 2.2 MHz. In some embodiments, the resonant frequency may be about 1.2 to 2.8 MHz, 1.4 to 2.6 MHz, 1.6 to 2.4 MHz, or 1.8 to 2.2 MHz. In some embodiments, the resistance range at resonance may be at least about 0.1 kΩ, 0.2 kΩ, or 0.3 kΩ. In some embodiments, the resistance range at resonance may be at most about 1.7 kΩ, 1.5 kΩ, 1.3 kΩ, or 1.1 kΩ. In some embodiments, the resistance range at resonance may be about 0.1 to 1.7 kΩ, 0.2 to 1.5 kΩ, 0.3 to 1.3 kΩ, or 0.3 to 1.1 kΩ.

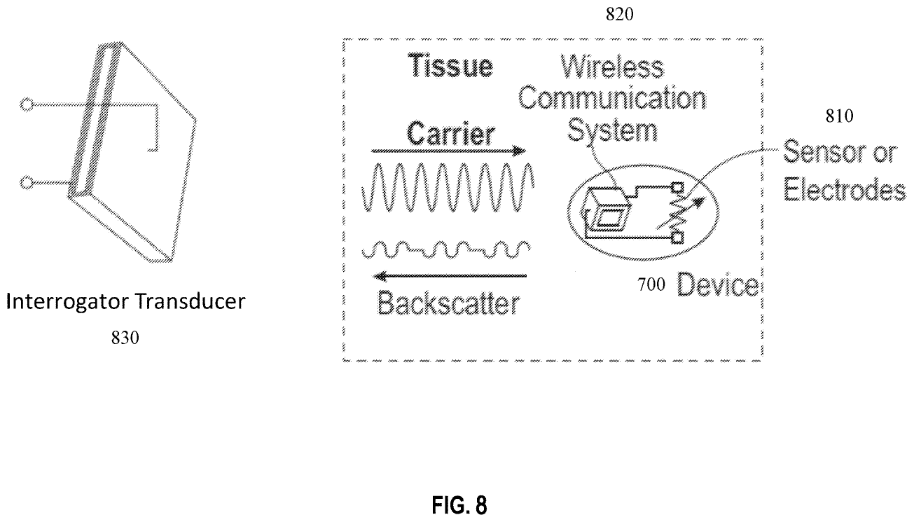

FIG. 8 shows a schematic of an exemplary device 700 having one or more sensors 810 and a wireless communication system 820. The sensors or electrodes 810 may be configured to electrically communicate with the wireless communication system 820. Additionally, the wireless communication system 820 may be configured to communicate with an external device having a communication system. For example, the external device may be an interrogator 830 having a communication system that includes one or more ultrasonic transducers.

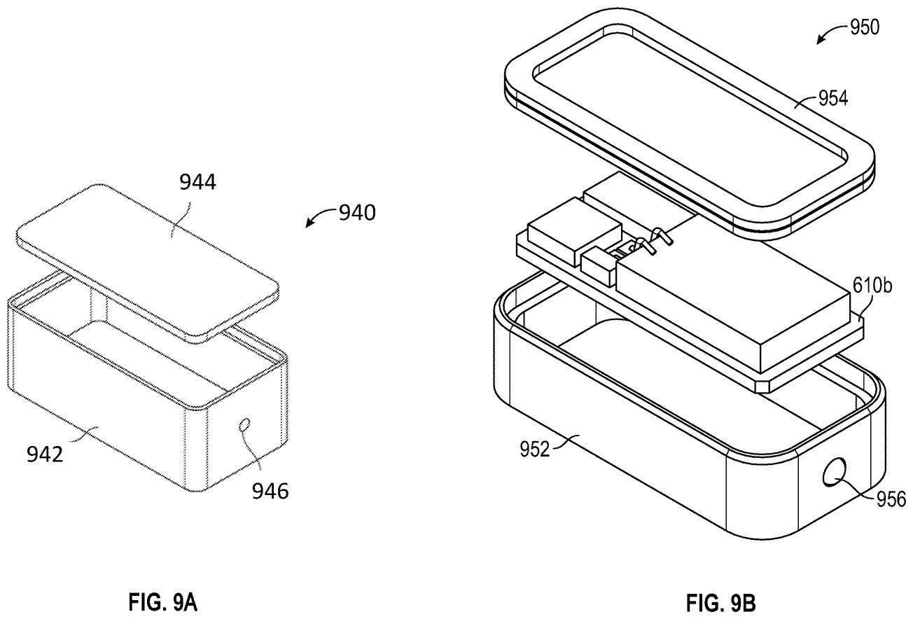

In some embodiments, the housing may house the wireless communication system, the one or more sensors, and the integrated circuit. The housing of the device can include a base, one or more sidewalls, and a top for enclosing the internal components of the device. In some embodiments, the housing may be at most about 0.25 mm high, 0.5 mm high, 1 mm high, or 2 mm high. In some embodiments, the housing may be at most 1 mm wide, 2 mm wide, or 3 mm wide. In some embodiments, the housing may be at most 1 mm long, 2 mm long, 3 mm long, 4 mm long, or 5 mm long. FIG. 9A shows an exploded view of an exemplary housing 940, according to some embodiments. The housing is made from a bioinert material, such as a bioinert metal (e.g., steel or titanium) or a bioinert ceramic (e.g., titania or alumina). In some embodiments, the housing may have no sharp corners or edges that could cause excessive reaction or inflammation beyond that caused by an implanting procedure. The housing is preferably hermetically sealed, which prevents body fluids from entering the body. In some embodiments, the hermetic seal may meet or exceed an equivalent leak rate of at least 2×10⁻⁸ atm-cc/sec Air, 5×10⁻⁸ atm-cc/sec Air, or 8×10⁻⁸ atm-cc/sec Air. The hermetically sealed housing may withstand shock, thermal cycling, and pressure change specifications identified by standards such as ISO 14708-1.

In some embodiments, the housing can include an acoustic window that serves at least one or both of the following: 1) it allows ultrasonic waves to penetrate the window and power the piezoelectric crystal of the device, and 2) it provides a compliant membrane that allows changes in intraocular pressure to transfer to the MEMS pressure sensor. In this way, the acoustic window allows ultrasonic waves to penetrate and equilibrate pressure external and internal to the housing. In some embodiments, the acoustic window may have a compliance that is at least about 400 times, 600 times, or 800 times larger than the compliance of a pressure sensor membrane of the pressure sensor. In some embodiments, the acoustic window may have a compliance that is at most about 1600 times, 1400 times, or 1,200 times larger than the compliance of a pressure sensor membrane of the pressure sensor. In some embodiments, the acoustic window may have a compliance that is at most about 400 to 1600 times, 600 to 1400 times, or 800 to 1,200 times larger than the compliance of a pressure sensor membrane of the pressure sensor. In some embodiments, the acoustic window may be oriented anterior to the Coronal Plane. The equilibration of pressure enables accurate IOP measurements while protecting the sensor within the housing. For example, the top 944 of the housing 940 can include an acoustic window. An acoustic window is a thinner material (such as a foil) that allows acoustic waves to penetrate the housing 940 so that they may be received by one or more ultrasonic transducers within the body of the device. In some embodiments, the housing (or the acoustic window of the housing) may be thin to allow ultrasonic waves to penetrate through the housing. In some embodiments, the thickness of the housing (or the acoustic window of the housing) is about 100 micrometers (μm) or less in thickness, such as about 75 μm or less, about 50 μm or less, about 25 μm or less, about 15 μm or less, or about 10 μm or less. In some embodiments, the thickness of the housing (or the acoustic window of the housing) is about 5 μm to about 10 μm, about 10 μm to about 15 μm, about 15 μm to about 25 μm, about 25 μm to about 50 μm, about 50 μm to about 75 μm, or about 75 μm to about 100 μm in thickness. In some embodiments, the acoustic window can be made from a metallic film.

The housing of the device is relatively small, which allows for comfortable and long-term implantation while limiting tissue inflammation that is often associated with implanting devices. In some embodiments, the longest dimension of the housing of the device is about 8 mm or less, about 7 mm or less, about 6 m or less, about 5 mm or less, about 4 mm or less, about 3 mm or less, about 2 mm or less, about 1 mm or less, about 0.5 mm or less, about 0.3 mm or less, about 0.1 mm or less in length. In some embodiments, the longest dimension of the housing of the device is about 0.05 mm or longer, about 0.1 mm or longer, about 0.3 mm or longer, about 0.5 mm or longer, about 1 mm or longer, about 2 mm or longer, about 3 mm or longer, about 4 mm or longer, about 5 mm or longer, about 6 mm or longer, or about 7 mm or longer in the longest dimension of the device. In some embodiments, the longest dimension of the housing of the device is about 0.3 mm to about 8 mm in length, about 1 mm to about 7 mm in length, about 2 mm to about 6 mm in length, or about 3 mm to about 5 mm in length. In some embodiments, the housing of the implantable device has a volume of about 10 mm³ or less (such as about 8 mm³ or less, 6 mm³ or less, 4 mm³ or less, or 3 mm³ or less). In some embodiments, the housing of the implantable device has a volume of about 0.5 mm³ to about 8 mm³, about 1 mm³ to about 7 mm³, about 2 mm³ to about 6 mm³, or about 3 mm³ to about 5 mm³.

The housing may be filled with an acoustic medium and void of water, moisture, or air bubbles. The acoustic medium may have a density that avoids an impedance mismatch with surrounding tissue. The acoustic medium may be electrically non-conductive. For example, the housing 940 may be filled with a polymer or oil (such as a silicone oil). The material can fill empty space within the housing to reduce acoustic impedance mismatch between the tissue outside of the housing and within the housing. Accordingly, an interior of the device is preferably void of air or vacuum. A port can be included on the housing, for example one of the sidewalls 942 of housing 940, there may be a port 946 to allow the housing to be filled with the acoustic medium. Once the housing 940 is filled with the material, the port 946 can be sealed to avoid leakage of the material after implantation.

FIG. 9B shows an exploded view of exemplary housing 950 that shows the housing is configured to house the circuit board 610b, according to some embodiments. Similar to housing 940, the housing 950 includes sidewalls 952, port 956, and a top 954.

In some embodiments, the housing 940, 950 may include externally attached features that allow placement and fixation of the device within or on an eye. The externally attached features do not interfere with ultrasound transmission, pressure transmission, or mounting of the device within or on the eye. For example, the housing may have externally attached features which allow placement and fixation into the lens capsule of the eye without interfering with the patient's line of sight or intraocular lens placement (if applicable). In some embodiments, the externally attached features may be free of sharp corners or edges that could cause excessive reaction or inflammation beyond that caused by the mounting procedure, or rough surfaces which are not required for the correct functioning of the device. In some embodiments, any externally attached features may not increase the rigid dimensions of the implant by more than 0.50 mm in height, 1.00 mm in width, or 1.50 mm in length.

### Interrogator

In some embodiments, the device may be configured to wirelessly communicate with components external to the device for IOP measuring operations. For example, the device may be configured to wirelessly communicate with an external interrogator. Through the wireless communication, the interrogator may be configured to instruct the device to collect a plurality of IOP measurements. The external interrogator may include one or more transducers, one or more sensors, and one or more force gauges.

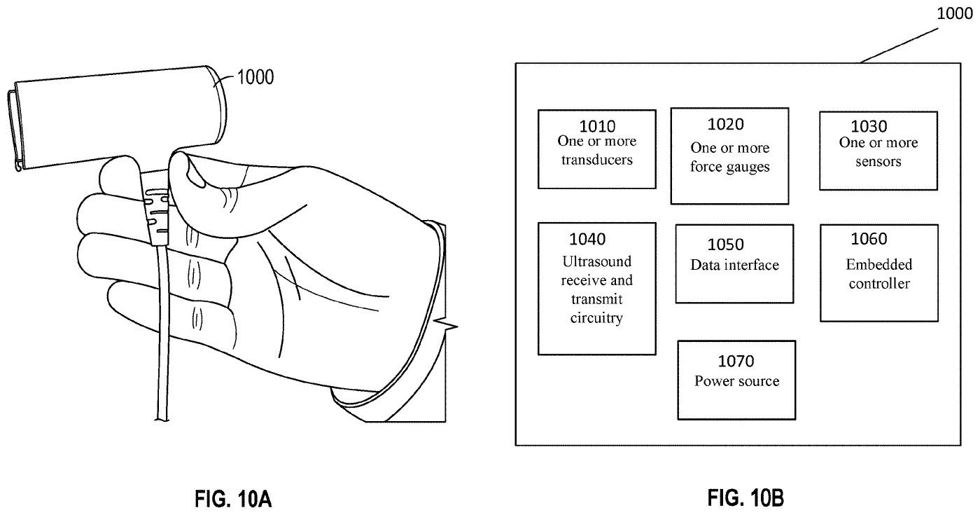

An exemplary interrogator 1000 is shown in FIG. 10A, according to some embodiments. An exemplary schematic of the exemplary interrogator 1000 is shown in FIG. 10B, according to some embodiments. The interrogator of FIG. 10A-B may be configured to wirelessly communicate with devices such as devices 300, 400, and 500. The interrogator 1000 may include one or more transducers 1010 for wireless communication, one or more force gauges 1020 for measuring force applied by the interrogator, and one or more sensors 1030 for measuring ambient conditions. In some embodiments, the one or more transducers 1010 may include an ultrasonic transducer. The ultrasonic transducer may be configured to ultrasonically couple to skin of an eyelid, skin over a brow bone, skin over a nasal bone, or skin over an eye socket to facilitate ultrasonic communication between the interrogator and the device mounted on or within an eye. In some embodiments, an ultrasound coupling gel or an alternative couplant may be used to ultrasonically couple the interrogator to the skin.

Ultrasonically coupling the ultrasonic transducer to the skin includes applying a contact force by the interrogator on the skin. Since such an applied contact force may adversely affect IOP measurements from the device, it is preferable to use a minimum amount of contact force for a more accurate TOP measurement. In some embodiments, the interrogator may include a force gauge configured to measure a force applied on the skin by the interrogator. For example, the interrogator 1000 may include one or more force gauges 1020 for this purpose. In some embodiments, the interrogator is configured to operate the device to determine a plurality of IOP measurements as the force gauge measures a decreasing force. The plurality of IOP measurements may be matched to corresponding gauge measurements to determine the IOP measurement collected at the lowest measured force.

In some embodiments, the interrogator includes one or more sensors configured to measure ambient conditions. For example, interrogator 1000 may include one or more sensors 1030 as shown in FIG. 10. The one or more sensors of the interrogator may include a pressure sensor for measuring ambient pressure. Optionally, the interrogator may further include a temperature sensor for measuring ambient temperature, which can be used to calibrate the pressure sensor used for measuring ambient pressure. The interrogator 1000 may be configured to receive the IOP measurements collected by the one or more sensors (such as one or more sensors 608) of the device (such as devices 100, 300, 400, 500), measure ambient conditions via the one or more sensors 1030 of the interrogator 1000, determine a final IOP reading by compensating (as necessary) the IOP measurements with ambient measurements, and communicate the final IOP measurement to a recipient external to both the interrogator and the device. In some embodiments, the interrogator may compensate the IOP measurements based on differences between the measured IOP and the measured ambient pressure. Since the difference between the IOP and ambient pressure is a biologically relevant value, in some embodiments, the compensation may simply be the difference between the IOP and ambient pressure. In some embodiments, the interrogator may compensate the IOP measurements using measured ambient pressure and a measured temperature inside the eye.

In some embodiments, the interrogator 1000 may include ultrasound receive and transmit circuitry 1040, a data interface 1050, an embedded controller 1060, and a power source 1070. In some embodiments, the device may be configured to rely on power transmission from the external interrogator. The power transmission from the interrogator may be used to power the device to initiate IOP measurements collected by the one or more sensors of the device. In some embodiments, the ultrasonic transducer of the interrogator may be configured to transmit instructions to the device. The instructions from the interrogator may instruct the device to reset itself, enter a specific mode, set device parameters, or begin a transmission sequence.

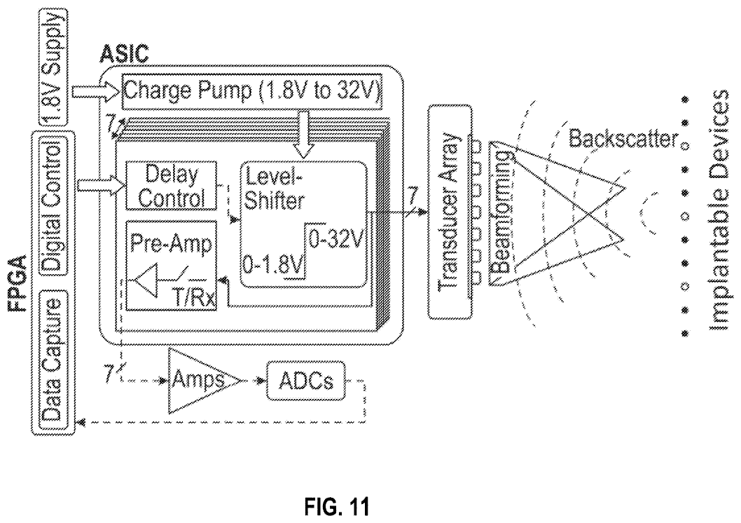

An exemplary interrogator is shown in FIG. 11, according to some embodiments. The illustrated interrogator shows a transducer array with a plurality of ultrasonic transducers. In some embodiments, the transducer array includes 1 or more, 2 or more, 3 or more, 5 or more, 7 or more, 10 or more, 15 or more, 20 or more, 25 or more, 50 or more, 100 or more 250 or more, 500 or more, 1000 or more, 2500 or more, 5000 or more, or 10,000 or more transducers. In some embodiments, the transducer array includes 100.000 or fewer, 50,000 or fewer, 25,000 or fewer, 10,000 or fewer, 5000 or fewer, 2500 or fewer, 1000 or fewer, 500 or fewer, 200 or fewer, 150 or fewer, 100 or fewer, 90 or fewer, 80 or fewer, 70 or fewer, 60 or fewer, 50 or fewer, 40 or fewer, 30 or fewer, 25 or fewer, 20 or fewer, 15 or fewer, 10 or fewer, 7 or fewer or 5 or fewer transducers. The transducer array can be, for example a chip comprising 50 or more ultrasonic transducer pixels.

The interrogator shown in FIG. 11 illustrates a single transducer array; however the interrogator can include 1 or more, 2 or more, or 3 or more separate arrays. In some embodiments, the interrogator includes 10 or fewer transducer arrays (such as 9, 8, 7, 6, 5, 4, 3, 2, or 1 transducer arrays). The separate arrays, for example, can be placed at different points of a subject, and can communicate to the same or different implantable devices. In some embodiments, the arrays are located on opposite sides of an implantable device. The interrogator can include an application specific integrated circuit (ASIC), which includes a channel for each transducer in the transducer array. In some embodiments, the channel includes a switch (indicated in FIG. 11 by “T/Rx”). The switch can alternatively configure the transducer connected to the channel to transmit ultrasonic waves or receive ultrasonic waves. The switch can isolate the ultrasound receiving circuit from the higher voltage ultrasound transmitting circuit.

In some embodiments, the transducer connected to the channel is configured only to receive or only to transmit ultrasonic waves, and the switch is optionally omitted from the channel. The channel can include a delay control, which operates to control the transmitted ultrasonic waves. The delay control can control, for example, the phase shift, time delay, pulse frequency and/or wave shape (including amplitude and wavelength). The delay control can be connected to a level shifter, which shifts input pulses from the delay control to a higher voltage used by the transducer to transmit the ultrasonic waves. In some embodiments, the data representing the wave shape and frequency for each channel can be stored in a ‘wave table’. This allows the transmit waveform on each channel to be different. Then, delay control and level shifters can be used to ‘stream’ out this data to the actual transmit signals to the transducer array. In some embodiments, the transmit waveform for each channel can be produced directly by a high-speed serial output of a microcontroller or other digital system and sent to the transducer element through a level shifter or high-voltage amplifier. In some embodiments, the ASIC includes a charge pump (illustrated in FIG. 11) to convert a first voltage supplied to the ASIC to a higher second voltage, which is applied to the channel. The channels can be controlled by a controller, such as a digital controller, which operates the delay control.

In the ultrasound receiving circuit, the received ultrasonic waves are converted to current by the transducers (set in a receiving mode), which is transmitted to a data capture circuit. In some embodiments, an amplifier, an analog-to-digital converter (ADC), a variable-gain-amplifier, or a time-gain-controlled variable-gain-amplifier which compensates for tissue loss, and/or a band pass filter is included in the receiving circuit. The ASIC can draw power from a power supply, such as a battery (which is preferred for a wearable embodiment of the interrogator). In the embodiment illustrated in FIG. 11, a 1.8V supply is provided to the ASIC, which is increased by the charge pump to 32V, although any suitable voltage can be used. In some embodiments, the interrogator includes a processor and or a non-transitory computer readable memory. In some embodiments, the channel described above does not include a T/Rx switch but instead contains independent Tx (transmit) and Rx (receive) with a high-voltage Rx (receiver circuit) in the form of a low noise amplifier with good saturation recovery. In some embodiments, the T/Rx circuit includes a circulator. In some embodiments, the transducer array contains more transducer elements than processing channels in the interrogator transmit/receive circuitry, with a multiplexer choosing different sets of transmitting elements for each pulse. For example, 64 transmit receive channels connected via a 3:1 multiplexer to 192 physical transducer elements—with only 64 transducer elements active on a given pulse.

In some embodiments, the interrogator is an external device (i.e., not implanted, but may be attached or held to an outer bodily surface). By way of example, the external interrogator can be a handheld interrogator (such as a wand), which may be a held by a user (such as the patient having the device implanted or mounted within or on her/his eye, or another person). The user may move the handheld external interrogator towards the eye having the implanted/mounted device to operate the implanted/mounted device. For example, the handheld interrogator may be placed on skin of an eyelid, skin over a brow bone, skin over a nasal bone, or skin over an eye socket to operate the implanted/mounted device to take the one or more measurements of IOP. In some embodiments, aiming the external interrogator towards the implanted/mounted device operates the device to take one or more measurements of IOP. In some embodiments, the handheld interrogator may operate the implanted/mounted device one or more times per day (such as 2-3 per day).

Physical contact between the eye/eyelid of a patient and the interrogator enables the interrogator to receive measurements from the implanted/mounted device. In some embodiments, the interrogator may be physically fixed (not sutured or implanted) to a patient. For example, the interrogator may be fixed to a patient's face or patient's skin surrounding the eye having the implanted/mounted device via a strap, or the like. Skin surrounding the eye may include, skin of an eyelid, skin over a brow bone, skin over a nasal bone, or skin over an eye socket. Fixing the interrogator to the patient allows the interrogator to continuously monitor TOP without requiring the patient or another user to hold the device in place. The fixed interrogator may be configured to run a program designed to activate the implanted/mounted device to take a measurement over time. In some embodiments, the fixed interrogator may be used to monitor IOP while a patient sleeps.

The specific design of the transducer array depends on the desired penetration depth, aperture size, and size of the individual transducers within the array. The Rayleigh distance, R, of the transducer array is computed as:

$$R = \frac{{D}^{2} - {λ}^{2}}{4 λ} \approx \frac{{D}^{2}}{4 λ} , {D}^{2} ≫ {λ}^{2}$$

where D is the size of the aperture and λ is the wavelength of ultrasound in the propagation medium. As understood in the art, the Rayleigh distance is the distance at which the beam radiated by the array is fully formed. That is, the pressure filed converges to a natural focus at the Rayleigh distance in order to maximize the received power. Therefore, in some embodiments, the implantable device is approximately the same distance from the transducer array as the Rayleigh distance.

The individual transducers in a transducer array can be modulated to control the Raleigh distance and the position of the beam of ultrasonic waves emitted by the transducer array through a process of beamforming or beam steering. Techniques such as linearly constrained minimum variance (LCMV) beamforming can be used to communicate a plurality of implantable devices with an external ultrasonic transceiver. See, for example, Bertrand et al., Beamforming Approaches for Untethered, Ultrasonic Neural Dust Motes for Cortical Recording: a Simulation Study, IEEE EMBC (August 2014). In some embodiments, beam steering is performed by adjusting the power or phase of the ultrasonic waves emitted by the transducers in an array.

In some embodiments, the interrogator includes one or more of instructions for beam steering ultrasonic waves using one or more transducers, instructions for determining the relative location of one or more implantable devices, instructions for monitoring the relative movement of one or more implantable devices, instructions for recording the relative movement of one or more devices (such as devices 100, 300, 400, 500) mounted on or within an eye, and instructions for deconvoluting backscatter from a plurality of implantable devices.

Optionally, the interrogator is controlled using a separate computer system, such as a mobile device (e.g., a smartphone or a table). The computer system can wirelessly communicate to the interrogator, for example through a network connection, a radiofrequency (RF) connection, or Bluetooth. The computer system may, for example, turn on or off the interrogator or analyze information encoded in ultrasonic waves received by the interrogator.

### Ultrasonic Communication

The device and the interrogator wirelessly communicate with each other, for example using ultrasonic waves. The communication may be a one-way communication (for example, the interrogator transmitting information to the device, or the device transmitting information to the interrogator), or a two-way communication (for example, the interrogator transmitting information to the device, or the device transmitting information to the interrogator). Information transmitted from the device to the interrogator may rely on, for example, a backscatter communication protocol. For example, the interrogator may transmit ultrasonic waves to the device, which emits backscatter waves that encode the information. The interrogator can receive the backscatter waves and decipher the information encoded in the received backscatter waves.

In some embodiments, the one or more ultrasonic transducers of the device may include a piezoelectric crystal configured to receive commands from ultrasonic energy transmitted from the external interrogator. The device may decode pulse interval encoded commands transmitted from the external interrogator and may passively transmit data to the external interrogator via amplitude-modulated, backscatter communication. In some embodiments, the device receives ultrasonic waves from the interrogator through one or more ultrasonic transducers on the implantable device, and the received waves can encode instructions for operating the implantable device. For example, vibrations of the ultrasonic transducer(s) on the device generate a voltage across the electric terminals of the transducer, and current flows through the device, including the integrated circuit. The current (which may be generated, for example, using one or more ultrasonic transducers) can be used to charge an energy storage circuit, which can store energy to be used to emit an electrical pulse, for example after receiving a trigger signal. The trigger signal can be transmitted from the interrogator to the implantable device, signaling that an electrical pulse should be emitted. In some embodiments, the trigger signal includes information regarding the electrical pulse to be emitted, such as frequency, amplitude, pulse length, or pulse shape (e.g., alternating current, direct current, or pulse pattern). A digital circuit can decipher the trigger signal and operate the electrodes and electrical storage circuit to emit the pulse.

In some embodiments, ultrasonic backscatter is emitted from the device, which can encode information relating to the device. In some embodiments, a device is configured to detect a physiological condition describing IOP, and information regarding the detected physiological condition can be transmitted to the interrogator by the ultrasonic backscatter. To encode physiological condition in the backscatter, current flowing through the ultrasonic transducer(s) of the device is modulated as a function of the encoded information, such as a measured physiological condition. In some embodiments, modulation of the current can be an analog signal, which may be, for example, directly modulated by the detected physiological condition. In some embodiments, modulation of the current encodes a digitized signal, which may be controlled by a digital circuit in the integrated circuit. The backscatter is received by an external interrogator (which may be the same or different from the external interrogator that transmitted the initial ultrasonic waves). The information from the electrophysiological signal can thus be encoded by changes in amplitude, frequency, or phase of the backscattered ultrasound waves.

In some embodiments, the ultrasound communication does not raise the temperature of any part of the eye more than about 1.5° C. at any time, in accordance with ISO 14708-01:2014 clause 17 which stipulates any surface of the implant shall not exceed a temperature increase of 2° C.

In some embodiments, the ultrasound communication may be established when the piezoelectric crystal of the device is about 5 mm+/−20% distance from the interrogator head. In some embodiments, the ultrasound communication may be established when a surface of the piezoelectric crystal is at most about a 3 mm, 5 mm, 7 mm, or 9 mm distance from a surface of the interrogator configured to touch skin of an eyelid, skin over a brow bone, skin over a nasal bone, or skin over an eye socket. In some embodiments, the ultrasound communication may be established when a surface of the piezoelectric crystal is at least about 1 mm, 2 mm, or 3 mm distance from the interrogator configured to touch skin of an eyelid, skin over a brow bone, skin over a nasal bone, or skin over an eye socket. In some embodiments, the ultrasound communication may be established when a surface of the piezoelectric crystal is about 1-9 mm, 2-7 mm, or 3-5 mm distance from the interrogator configured to touch skin of an eyelid, skin over a brow bone, skin over a nasal bone, or skin over an eye socket. Once established, the ultrasound communication may tolerate typical involuntary eye movement for the brief duration of the IOP measurement.

FIG. 8 shows an interrogator in communication with an implantable device. The external ultrasonic transceiver emits ultrasonic waves (“carrier waves”), which can pass through tissue. The carrier waves cause mechanical vibrations on the ultrasonic transducer (e.g., a bulk piezoelectric transducer, a PUMT, or a CMUT). A voltage across the ultrasonic transducer is generated, which imparts a current flowing through an integrated circuit on the implantable device. The current flowing through to the ultrasonic transducer causes the transducer on the implantable device to emit backscatter ultrasonic waves. In some embodiments, the integrated circuit modulates the current flowing through the ultrasonic transducer to encode information, and the resulting ultrasonic backscatter waves encode the information. The backscatter waves can be detected by the interrogator, and can be analyzed to interpret information encoded in the ultrasonic backscatter.

The instructions from the interrogator to the device can be carried by the ultrasonic carrier. Specifically, the ultrasonic carrier generated by the ultrasonic transducer of the interrogator may include a series of ultrasonic pulses that have a varying number of carrier periods. The number of carrier periods encode information specific to the device. For example, based on the number of carrier periods, the information may include instructions for the device to begin a data transmission sequence. The transmission sequence can include steps for measuring IOP data and encoding the IOP data as ultrasonic backscatter. The encoding includes backscattering the IOP data on the ultrasonic carrier to modulate the electrical current and converting the modulated current to ultrasonic backscatter for transmission to the interrogator. The number of carrier periods may encode other information related to the device. For example, the information may include instructions for the device to reset itself, enter a specific mode, or set device parameters.

Communication between the interrogator and the implantable device can use a pulse-echo method of transmitting and receiving ultrasonic waves. In the pulse-echo method, the interrogator transmits a series of interrogation pulses at a predetermined frequency, and then receives backscatter echoes from the implanted device. In some embodiments, the pulses are square, rectangular, triangular, sawtooth, or sinusoidal. In some embodiments, the pulses output can be two-level (GND and POS), three-level (GND, NEG. POS), 5-level, or any other multiplelevel (for example, if using 24-bit DAC). In some embodiments, the pulses are continuously transmitted by the interrogator during operation. In some embodiments, when the pulses are continuously transmitted by the interrogator a portion of the transducers on the interrogator are configured to receive ultrasonic waves and a portion of the transducers on the interrogator are configured to transmit ultrasonic waves. Transducers configured to receive ultrasonic waves and transducers configured to transmit ultrasonic waves can be on the same transducer array or on different transducer arrays of the interrogator. In some embodiments, a transducer on the interrogator can be configured to alternatively transmit or receive the ultrasonic waves. For example, a transducer can cycle between transmitting one or more pulses and a pause period. The transducer is configured to transmit the ultrasonic waves when transmitting the one or more pulses, and can then switch to a receiving mode during the pause period.

In some embodiments, the backscattered waves are digitized by the implantable device. For example, the implantable device can include an oscilloscope or analog-to-digital converter (ADC) and/or a memory, which can digitally encode information in current (or impedance) fluctuations. The digitized current fluctuations, which can encode information, are received by wireless communication system, which then transmits digitized ultrasonic waves. The digitized data can compress the analog data, for example by using singular value decomposition (SVD) and least squares-based compression. In some embodiments, the compression is performed by a correlator or pattern detection algorithm. The backscatter signal may go through a series of non-linear transformation, such as 4th order Butterworth bandpass filter rectification integration of backscatter regions to generate a reconstruction data point at a single time instance. Such transformations can be done either in hardware (i.e., hard-coded) or in software.

In some embodiments, the digitized data can include a unique identifier. The unique identifier can be useful, for example, in a system comprising a plurality of implantable devices and/or an implantable device comprising a plurality of electrode pairs. For example, the unique identifier can identify the implantable device of origin when from a plurality of implantable devices, for example when transmitting information from the implantable device (such as a verification signal). The digitized circuit can encode a unique identifier to identify and/or verify which electrode pairs emitted the electrical pulse.

In some embodiments, the digitized signal compresses the size of the analog signal. The decreased size of the digitized signal can allow for more efficient reporting of information encoded in the backscatter. By compressing the size of the transmitted information through digitization, potentially overlapping signals can be accurately transmitted.

In some embodiments, an interrogator communicates with a plurality of devices. This can be performed, for example, using multiple-input, multiple output (MIMO) system theory. For example, communication between the interrogator and the plurality of implantable devices using time division multiplexing, spatial multiplexing, or frequency multiplexing. The interrogator can receive a combined backscatter from the plurality of the implantable devices, which can be deconvoluted, thereby extracting information from each implantable device. In some embodiments, interrogator focuses the ultrasonic waves transmitted from a transducer array to a particular implantable device through beamsteering. The interrogator focuses the transmitted ultrasonic waves to a first device, receives backscatter from the first device, focuses transmitted ultrasonic waves to a second device, and receives backscatter from the second device. In some embodiments, the interrogator transmits ultrasonic waves to a plurality of devices, and then receives ultrasonic waves from the plurality of devices.

The wireless communication system, which can communicate with a separate device (such as an external interrogator or another device). For example, the wireless communication 420 may be configured to receive instructions for emitting ultrasonic backscatter associated with measured IOP data from the one or more sensors. The wireless communication system can include, for example one or more ultrasonic transducers. The wireless communication system may also be configured to receive energy (for example, through ultrasonic waves) from another device, which can be used to power the implantable device.

In addition to providing the device with instructions, in some embodiments, the ultrasonic carrier from the interrogator may transmit vibrational energy configured to power the device. That is, the ultrasonic pulses of the ultrasonic carrier is delivered to the device at a frequency suitable for imparting energy to power the ASIC.

In some embodiments, the implantable device can also be operated to transmit information (i.e., uplink communication), which can be received by the interrogator, through the wireless communication system. In some embodiments, the wireless communication system is configured to actively generate a communication signal (e.g., ultrasonic waves) that encode the information. In some embodiments, the wireless communication system is configured to transmit information encoded on backscatter waves (e.g., ultrasonic backscatter waves). Backscatter communication provides a lower power method of transmitting information, which is particularly beneficial for small devices to minimize energy sues. By way of example, the wireless communication system may include one or more ultrasonic transducers configured to receive ultrasonic waves and emit an ultrasonic backscatter, which can encode information transmitted by the implantable device. Current flows through the ultrasonic transducer, which can be modulated to encode the information. The current may be modulated directly, for example by passing the current through a sensor that modulates the current, or indirectly, for example by modulating the current using a modulation circuit based on a detected physiological condition such as IOP.

The information wirelessly transmitted using the wireless communication system can be received by an interrogator. In some embodiments, the information is transmitted by being encoded in backscatter waves (e.g., ultrasonic backscatter). The backscatter can be received by the interrogator, for example, and deciphered to determine the encoded information. Additional details about backscatter communication are provided herein, and additional examples are provided in WO 2018/009905: WO 2018/009908; WO 2018/0091010: WO 2018/009911; WO 2018/009912; International Patent Application No. PCT/US2019/028381; International Patent Application No. PCT/US2019/028385; and International Patent Application No. PCT/2019/048647; each of which is incorporated herein by reference for all purposes. The information can be encoded by the integrated circuit using a modulation circuit. The modulation circuit is part of the wireless communication system, and can be operated by or contained within the integrated circuit.

Methods for Detecting Intraocular Pressure and/or Treating Eye Disease

The interrogator and device may be configured to enable on-demand IOP sensing. The interrogator may be configured to initiate a device mounted on or within an eye to measure IOP. Based on instructions from the interrogator, the device may take a plurality of IOP measurements and transmit the messages encoded with the IOP measurements to the interrogator. The interrogator may be configured to decode the message and adjust the IOP measurements based on an ambient pressure measured by the interrogator. The adjusted IOP measurement may be communicated to a recipient external to both the interrogator and the device.

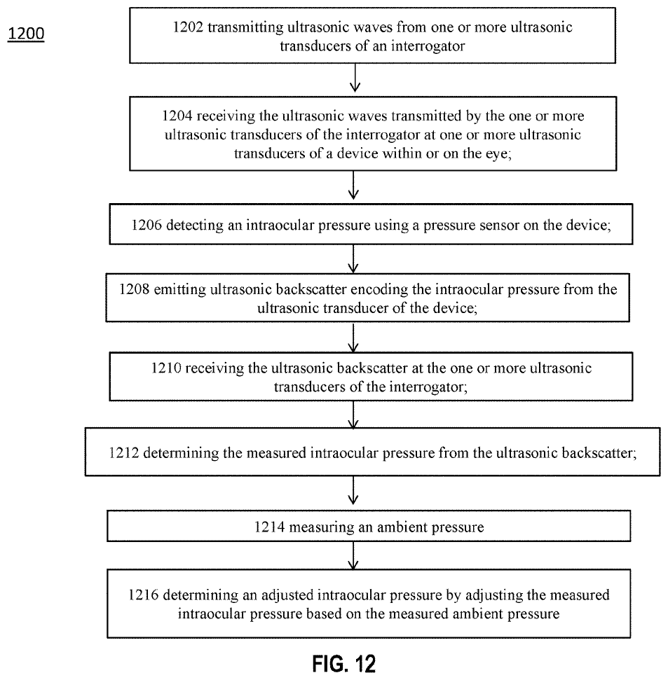

FIG. 12 is a flowchart demonstrating a method 1200 of measuring intraocular pressure of an eye. At step 1202, ultrasonic waves are transmitted from an interrogator to a device external to the interrogator. The device may be mounted on or within an eye. The interrogator and the device may each include one or more ultrasonic transducers to receive and transmit ultrasonic waves. At step 1204, the ultrasonic waves are received by one or more ultrasonic transducers of the device. The ultrasonic waves may operate the device to collect IOP measurements via a pressure sensor. At step 1206, IOP is detected via a pressure sensor on the device. In some embodiments, the device may collect two distinct values with each interrogation from the interrogator, one corresponding to the IOP measured from the pressure sensor and another corresponding to the intraocular temperature (IOT) from the temperature sensor. The temperature sensor data may be used for compensation purposes to increase accuracy of a final pressure measurement, for example by calibrating the pressure sensor. In some embodiments, the pressure sensor is calibrated using the measured temperature at the device, and the device communicates the calibrated temperature to the interrogator. In some embodiments, the measurements of the pressure sensor and temperature sensor may be completed if there is power available to the device to complete the measurements. In some embodiments, the detected IOP is encoded by the device as ultrasonic backscatter. In some embodiments, the detected IOP and IOT is encoded by the device as ultrasonic backscatter. At step 1208, the ultrasonic backscatter is emitted from the device. At step 1210, the ultrasonic backscatter is received by one or more ultrasonic transducers of the interrogator. At step 1212, the measured IOP is determined from the ultrasonic backscatter. In some embodiments, the interrogator decodes the ultrasonic backscatter to determine the measured IOP from the device. At step 1214, ambient pressure is measured by the interrogator. In some embodiments, the ambient pressure is pressure away from the body. At step 1216, an adjusted IOP is determined by adjusting the measured IOP based on the measured ambient pressure. In some embodiments, no adjustment is needed based on the measured ambient pressure, in which case the adjusted IOP equals the measured ambient pressure.

In some embodiments, to perform IOP measuring operations, the ultrasonic transducer of the interrogator may be placed over an eyelid of an eye aiming towards the device implanted within or mounted on the eye. In some embodiments, the interrogator is ultrasonically coupled to the skin of an eyelid, skin over a brow bone, skin over a nasal bone, or skin over an eye socket by applying a force by the interrogator to the skin. In some embodiments, to perform IOP measuring operations, the interrogator is contacted to the skin and then moved away from the skin until the contact is lost. While the interrogator is in contact with the skin, the interrogator instructs the device to measure a plurality of IOPs while the interrogator measures a plurality of force magnitudes applied to the skin by the interrogator. In some embodiments, the interrogator selects a final IOP measurement from the plurality of TOP measurements associated with a minimal force applied by the interrogator.

Regular monitoring of IOP can play a key role in monitoring and preventing eye disease related to high TOP, such as glaucoma or ocular hypertension. A high TOP for a given patient may be determined based on whether the measured IOP is above a threshold. The threshold may be based on one or more of IOP trends of the patient and standard IOP values. Thus, the threshold may vary from patient to patient. Regular monitoring of IOP can enable early detection of higher than normal IOP and allows the patient an opportunity to receive early treatment options for minimizing vision loss associated with high IOP.

In the event high IOP is detected, the patient may be eligible for an eye drop medication, or other therapeutic agent, to decrease IOP. An effective amount of the therapeutic agent can be administered to the patient to lower the intraocular pressure (e.g., an ocular antihypertensive). Depending on the patient and the eye condition, more than one type of eye drop may be used to decrease IOP. Therapeutic agents that can lower IOP include, for example, prostaglandins, cannabinoid, beta blockers, alpha-adrenergic agonists, carbonic anhydrase inhibitors, rho kinase inhibitors, and miotic of cholinergic agents. Exemplary therapeutic agents that can be used to treat glaucoma or ocular hypertension, or to lower intraocular pressure, include acetazolamide, apraclonidine, brimonidine (e.g, brimonidine tartrate), carbachol, echothiphate (e.g., echothiphate iodide), methazolamide, mitomycin, nadolol, pilocarpine, and timolol (or a mixture of brimonidine and timolol).

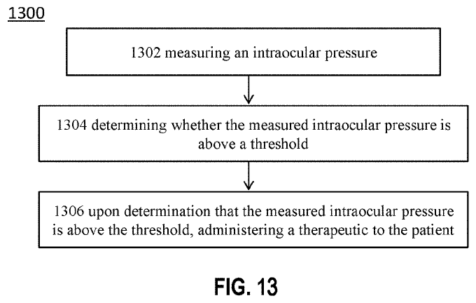

FIG. 13 is a flowchart demonstrating a method 1300 for treating a patient with an eye disease, such as glaucoma or ocular hypertension. At step 1302, IOP is measured. The IOP can be measured using, for example, a device (such as devices 12, 300, 400, 500) and an interrogator (such as interrogator 1000). The IOP measured may be a final IOP that is determined based on an initial IOP measured by device and an ambient pressure measured by the interrogator. At step 1304, the measured IOP is compared to a threshold. If the measured IOP is above the threshold, then the measured IOP is determined to be high. At step 1306, upon determination that the measured IOP is high, a therapeutic agent is administered to the patient to decrease IOP.

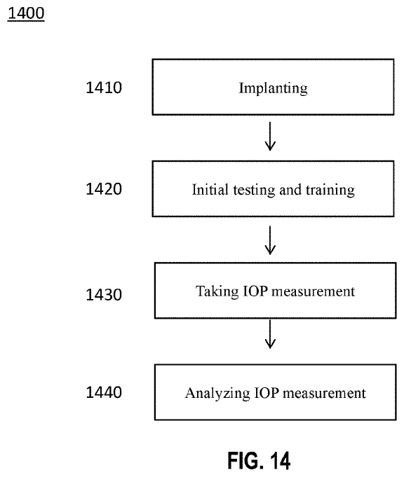

FIG. 14 is a flowchart demonstrating a method 1400 for using a device to monitor IOP of a patient, according to some embodiments. At step 1410, the device may be implanted in one of the patient's eyes during surgery. For example, the device may be implanted during surgery for intraocular lens placement. At step 1420, a first measurement is taken in presence of clinician. The patient may be instructed to measure IOP once a day. At step 1430, the patient will use an interrogator to take measurement as instructed. At step 1440, IOP measurements are uploaded onto a cloud and analyzed using a backend application. The physician can use this information to help the patient make more informed decisions about their treatment.

In some embodiments, the method 1400 may include a calibration step. The calibration may occur periodically after implantation, for example, to account for sensor reading drift. Calibration may involve recording IOP with a tonometer or alternate standard for measuring IOP. In some embodiments, the calibration may occur after a patient healing period, or if accuracy issues are suspected. In some embodiments, calibration may occur before implantation.

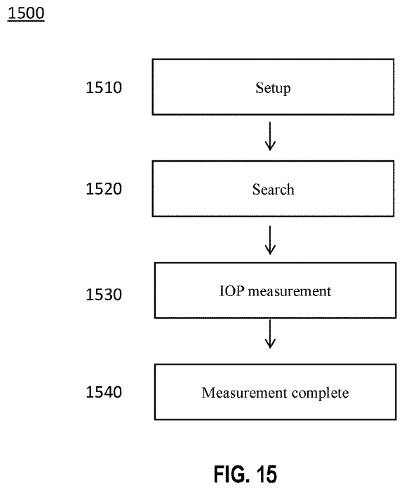

FIG. 15 is a flowchart demonstrating a method 1500 for taking IOP measurements with a device mounted on or within an eye of a patient and an external interrogator, according to some embodiments. The method 1500 may include a setup step 1510, a search step 1520, and an IOP measurement step 1550, and a completion step 1540. The method 1500 may take less than 2, 4, 6, 8, or 10 minutes. At step 1510, the interrogator is turned on and ultrasound coupling medium is placed on the interrogator tip or eyelid. At step 1520, the interrogator is placed against the patient's eyelid and moved until it has successful communication with the device. At step 1530, the device will take IOP measurement. At step 1540, the IOP measurement is complete.

### Exemplary Environmental Specifications

The device, packaging of the device, and methods of using the device comply with standard medical procedures. For example, the bioburden testing method of the device may comply with standard medical specifications, such as ISO 11737-1. Fluid and tissue contacting components of the device may, based upon the nature of body contact and contact duration, meet the requirements of EN ISO 10993-1. In some embodiments, the packaged device may be sterilized in accordance with ISO 11135 in order to reach a sterility assurance level (SAL) of at least 1/1,000,000 according to the requirement in EN 556. In some embodiments, the device may meet the Ethylene Oxide (EO) sterilization residual requirements according to ISO 10993-7. The device may withstand at least five cycles of EO sterilization without any physical damage or material degradation. The product's sterile packaging may retain the sterility of the device for a minimum of 1 year.

The device may be constructed to withstand the changes of pressure which can occur during transit or normal conditions of use. The device components shall withstand pressure changes without irreversible deformation, cracking or tearing due to absolute pressures of 70 kPa±3.5 kPa and 150 kPa±7.5 kPa applied for not less than 1 hour per ISO 14708-1. The device may be configured so that no irreversible change will be caused by the changes in temperature to which they can be subjected during transportation or storage. The device, in a sterile pack, may be subjected to a test in accordance with IEC 60068-2-14:2009, test Nb, where the low temperature value is −10° C.±3° C. and the high temperature value is 55° C.±2° C. The rate of change of temperature shall be 1° C./min±0.2° C./min. The device may be nonpyrogenic.

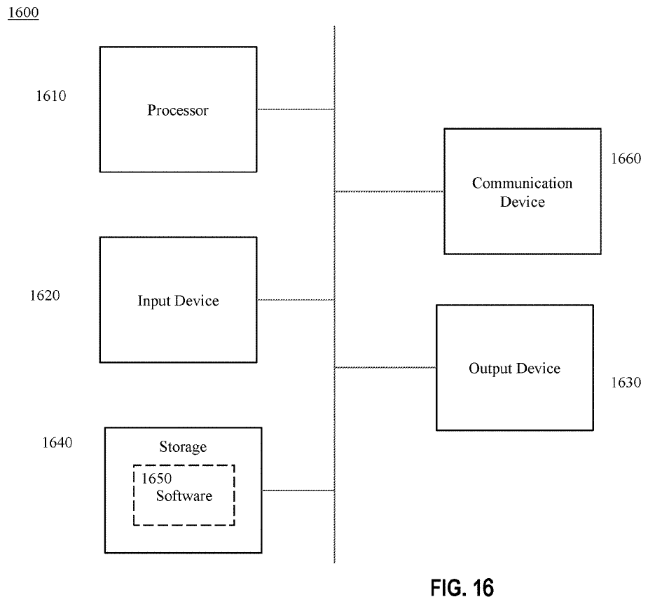

FIG. 16 illustrates an example of a computing device 1600 in accordance with some embodiments (such as for operating interrogator 14 of system 10), or a computing device for implementing methods 1200 and 1300 using the interrogator). Computing device 1600 can be a host computer connected to a network. Computing device 1600 can be a client computer or a server. As shown in FIG. 16, computing device 1600 can be any suitable type of microprocessor-based device, such as a personal computer, workstation, server, or handheld computing device (portable electronic device) such as a phone or tablet. The computing device 1600 can include, for example, one or more of processor 1610, input device 1620, output device 1630, storage 1640, and communication device 1660. Input device 1620 and output device 1630 can generally correspond to those described above and can either be connectable or integrated with the computer.

Input device 1620 can be any suitable device that provides input, such as a touch screen, keyboard or keypad, mouse, or voice-recognition device. Output device 1630 can be any suitable device that provides output, such as a touch screen, haptics device, or speaker.

Storage 1640 can be any suitable device that provides storage, such as an electrical, magnetic, or optical memory including a RAM, cache, hard drive, or removable storage disk. Communication device 1660 can include any suitable device capable of transmitting and receiving signals over a network, such as a network interface chip or device. The components of the computer can be connected in any suitable manner, such as via a physical bus or wirelessly.

Software 1650, which can be stored in storage 1640 and executed by processor 1610, can include, for example, the programming that embodies the functionality of the present disclosure (e.g., as embodied in the devices as described above).

Software 1650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1640, that can contain or store programming for use by or in connection with an instruction execution system, apparatus, or device.

Software 1650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic, or infrared wired or wireless propagation medium.

Computing device 1600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks. DSL, or telephone lines.

Computing device 1600 can implement any operating system suitable for operating on the network. Software 1650 can be written in any suitable programming language, such as C. C++, Java, or Python. In various embodiments, application software embodying the functionality of the present disclosure can be deployed in different configurations, such as in a client/server arrangement or through a Web browser as a Web-based application or Web service, for example.

In some embodiments, the computing device 1600 may store system configuration data and system calibration data. The computing device 1600 may also store and be able to report to the user the serial number and software and firmware versions for the interrogator. The computing device 1600 may have an event log. The computing device 1600 may monitor fault conditions. Fault conditions are any state where the system is unable to perform in accordance to product specifications.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The disclosures of all publications, patents, and patent applications referred to herein are each hereby incorporated by reference in their entireties. To the extent that any reference incorporated by reference conflicts with the instant disclosure, the instant disclosure shall control.

1. A device for measuring an intraocular pressure, comprising:
a pressure sensor configured to measure the intraocular pressure;
an ultrasonic transducer electrically coupled to the pressure sensor and configured to receive ultrasonic waves and emit ultrasonic backscatter encoding a pressure measured by the pressure sensor, and
a substrate attached to the pressure sensor and the ultrasonic transducer, and configured to interface a surface on or within an eye.

2. The device of claim 1, wherein the substrate has a partial or full ring structure.

3. The device of claim 1 or 2, wherein the substrate is configured to apply a force to the surface.

4. The device of claim 3, wherein the force applied by the substrate to the surface is a radial outward force.

5. The device of any one of claims 1-4, when the device is configured to be implanted within a capsular bag of the eye.

6. The device of any one of claims 1-5, wherein the substrate comprises one or more apertures configured to secure a surgical tool for guiding the device during implantation.

7. The device of any one of claims 1-6, comprising a housing configured to enclose the pressure sensor and the ultrasonic transducer.

8. The device of claim 7, wherein the housing is mounted on the substrate.

9. The device of claim 7 or 8, wherein the substrate has a partial or full ring structure, and comprises a mount configured to mount the housing.

10. The device of claim 9, wherein the mount extends radially inwardly or radially outwardly from the substrate.

11. The device of any one of claims 7-10, wherein the housing is hermetically sealed.

12. The device of any one of claims 7-11, wherein the housing comprises an acoustic window.

13. The device of claim 12, wherein the pressure sensor is positioned within the housing, and the acoustic window is configured to equilibrate a pressure inside the housing to a pressure outside the housing.

14. The device of any one of claims 7-13, wherein the housing is filled with a liquid or gel configured to transmit ultrasonic waves.

15. The device of claim 14, wherein the housing is filled with silicone oil.

16. The device of any one of the preceding claims, comprising a temperature sensor.

17. The device of claim 16, wherein the device is configured to calibrate the pressure measured by the pressure sensor using an eye temperature measured by the temperature sensor.

18. The device of any one of the preceding claims, wherein the ultrasonic transducer has a longest length dimension of 1 mm or less.

19. The device of any one of the preceding claims, wherein the surface comprises a capsular bag, haptics of an intraocular lens, or a contact lens.

20. The device of any one of the preceding claims, wherein the surface comprises an ins.

21. The device of any one of the preceding claims, wherein the surface comprises a lens capsule, an episclera, or on or near a pars plana of the eye.

22. The device of any one of the preceding claims, wherein the substrate comprises one or more fasteners for attaching the substrate to the surface of the eye.

23. The device of claim 22, comprising at least two fasteners positioned at opposite ends of the substrate.

24. The device of claim 22 or 23, wherein the fasteners comprise lateral hooks configured to attach to eye tissue.

25. The device of any one of claims 22-24, wherein the fasteners comprise vertical hooks configured to enter eye tissue.

26. The device of any one of the preceding claim, wherein the ultrasonic transducer is configured to receive ultrasonic waves that power the implantable device.

27. The device of any one of the preceding claim, wherein the ultrasonic waves are transmitted by an interrogator external to the device.

28. The device of any one of the preceding claim, comprising an integrated circuit in electrical communication with the pressure sensor and the ultrasonic transducer.

29. The device of claim 28, wherein the integrated circuit is configured to power the pressure sensor.

30. The device of claim 28 or 29, wherein the integrated circuit is configured to encode the measured pressure in the ultrasonic backscatter.

31. The device of any one of claims 28-30, wherein the housing encloses the integrated circuit.

32. The device of any one of claims 28-31, wherein the integrated circuit is coupled to a power circuit comprising a capacitor.

33. The device of claim 32, wherein the ultrasonic transducer is configured to receive ultrasonic waves that are converted into an electrical energy, which is stored by the power circuit.

34. The device of any one of claims 28-33, wherein the integrated circuit is configured to selectively operate the device in a communication mode or power storage mode.

35. The device of any one of the preceding claim, wherein the ultrasonic transducer is a piezoelectric crystal.

36. The device of any one of the preceding claim, wherein the device is configured to be implanted within the eye of a subject.

37. The device of claim 36, wherein the device is configured to be implanted within an anterior chamber of the eye.

38. The device of any one of the preceding claim, wherein the device is configured to be battery-less.

39. A system for measuring intraocular pressure of an eye, the system comprising:
the device of any one of claims 1-38; and
an interrogator comprising:
a pressure sensor configured to measure ambient pressure; and
one or more ultrasonic transducers configured to transmit the ultrasonic waves to implantable device, and receive the ultrasonic backscatter from the implantable device.

40. The system of claim 39, wherein the interrogator is configured to determine the measured intraocular pressure using the received ultrasonic backscatter.

41. The system of claim 40, wherein the interrogator is configured to determine an adjusted intraocular pressure by adjusting the measured intraocular pressure based on the measured ambient pressure.

42. The system of any one of claims 39-41, wherein the interrogator comprises a temperature configured to measure an ambient temperature.

43. The system of claim 42, wherein the interrogator is configured to calibrate the measured ambient pressure using the measured ambient temperature.

44. The system of any one of claims 39-43, wherein the interrogator is configured to calibrate the measured intraocular pressure using the eye temperature measured by the device.

45. The system of any one of claims 39-44, wherein the interrogator comprises a force gauge configured to measure a force applied by the interrogator.

46. The system of claim 45, wherein the interrogator is configured to operate the device to determine a plurality of IOP measurements as the force gauge measures a decreasing force.

47. The system of claim 46, wherein the interrogator is configured to select an IOP measurement at a lowest measured force.

48. The system of any one of claims 39-47, wherein the ultrasonic transducer of the interrogator is configured to transmit ultrasonic waves that power the implantable device.

49. A system for measuring intraocular pressure of an eye, comprising an interrogator comprising:
a pressure sensor configured to measure ambient pressure; and
one or more ultrasonic transducers configured to transmit the ultrasonic waves and receive the ultrasonic backscatter encoding an intraocular pressure measured by a device on or in the eye;
wherein the interrogator is configured to determine a measured intraocular pressure based on the received ultrasonic backscatter, and determine an adjusted intraocular pressure by adjusting the measured intraocular pressure based on the measured ambient pressure.

50. The system of claim 49, wherein the ultrasonic waves are configured to power the device.

51. The system of claim 49 or 50, wherein the ultrasonic waves are configured to encode instructions for one or more of resetting and the device, designating a mode of operation for the device, setting device parameters for the device, and beginning a data transmission sequence from the device.

52. A method of measuring intraocular pressure of an eye, comprising:
transmitting ultrasonic waves from one or more ultrasonic transducers of an interrogator;
receiving the ultrasonic waves transmitted by the one or more ultrasonic transducers of the interrogator at one or more ultrasonic transducers of a device within or on the eye;
detecting an intraocular pressure using a pressure sensor on the device;
emitting ultrasonic backscatter encoding the intraocular pressure from the ultrasonic transducer of the device;
receiving the ultrasonic backscatter at the one or more ultrasonic transducers of the interrogator;
determining the measured intraocular pressure from the ultrasonic backscatter;
measuring an ambient pressure; and
determining an adjusted intraocular pressure by adjusting the measured intraocular pressure based on the measured ambient pressure.

53. The method of claim 52, wherein the device is implanted in a capsular bag of the eye.

54. The method of claim 52 or 53, comprising converting energy from the ultrasonic waves into an electrical energy that powers the device.

55. The method of any one of claims 52-54, comprising instructing the device by the interrogator to execute one or more of resetting the device, designating a mode of operation of the device, setting parameters of the device, and beginning a data transmission sequence from the device.

56. The method of any one of claims 52-55, wherein pressure detection and measurement is configured to occur during a time in which no ultrasonic waves are being transmitted.

57. The method of any one of claims 52-56, comprising coupling the one or more ultrasonic transducers of the interrogator to an eyelid of the eye via a couplant.

58. The method of any one of claims 52-57, comprising applying a force by the interrogator to contact skin of an eyelid, skin over a brow bone, skin over a nasal bone, or skin over an eye socket, moving the interrogator away from the skin until contact with the skin is lost, and measuring by the interrogator a plurality of force magnitudes while the interrogator is in contact with the skin.

59. The method of claim 58, comprising receiving by the interrogator a plurality of intraocular pressure measurements while measuring the plurality force magnitudes.

60. The method of claim 59, comprising selecting from the plurality of intraocular pressure measurements a final intraocular pressure associated with a minimal force applied by the interrogator.

61. The method of any one of claims 52-60, comprising placing the ultrasonic transducer of the interrogator over an eyelid of the eye aiming towards the device.

62. The method of any one of claims 52-61, comprising placing the ultrasonic transducer of the interrogator over skin of an eyelid, skin over a brow bone, skin over a nasal bone, or skin over eye socket.

63. The method of any one of claims 52-62, comprising detecting an intraocular eye temperature, and calibrating the intraocular pressure detected by the device using the detected intraocular eye temperature.

64. The method of claim 63, wherein the intraocular temperature is encoded in the emitted ultrasonic backscatter, and the intraocular pressure detected by the device is calibrated by the interrogator.

65. The method of claim 63, wherein the intraocular pressure detected by the device is calibrated by the device.

66. A method for treating a patient with an eye disease, comprising:
measuring an intraocular pressure using a system of any one of claims 39-51;
determining whether the measured intraocular pressure is above a threshold; and
upon determination that the measured intraocular pressure is above the threshold, administering a therapeutic agent to the patient.

67. The method of claim 66, wherein the eye disease is glaucoma or ocular hypertension.

68. The method of claim 66 or 67, wherein the therapeutic agent decreases the intraocular pressure.

69. The method of any one of claims 66-68, wherein the threshold is determined based at least in part on routine measurements of the intraocular pressure.
